(19) United States
(12) Patent Application Publication
(10) Pub. No.: US 2024/0203534 A1
(43) Pub. Date: Jun. 20, 2024

(54) AGGREGATING GENOME DATA INTO BINS WITH SUMMARY DATA AT VARIOUS LEVELS

(71) Applicant: Illumina, Inc., San Diego, CA (US)

(72) Inventors: Andrew Warren, La Costa, CA (US); Benjamin Rinvelt, Madison, WI (US); Max Arseneault, Redondo Beach, CA (US)

(73) Assignee: Illumina, Inc., San Diego, CA (US)

(21) Appl. No.: 18/391,014

(22) Filed: Dec. 20, 2023

Related U.S. Application Data
(60) Provisional application No. 63/433,863, filed on Dec. 20, 2022.

(51) Int. Cl. G16B 50/10; G16B 30/00

(52) U.S. Cl. CPC G16B 50/10; G16B 30/00

(57) ABSTRACT

Systems, methods, and apparatus are described herein for aggregating genome data into bins with summary data at various levels. As described herein, a computing device may be configured to receive genome data associated with a genome. The computing device may be configured to generate an aggregate file using the received genome data. The aggregate file may include a plurality of bins at a plurality of depths. The computing device may be configured to determine summary data for respective reads associated with one or more respective portions of the genome covered by respective bins of the plurality of bins. The computing device may be configured to store the summary data for the respective reads in respective bins of the plurality of bins. The computing device may be configured to display a portion of the summary data in response to a selection of a genomic region by a user.

### CROSS-REFERENCE TO RELATED APPLICATIONS

This application claims the benefit of U.S. Provisional Patent Application No. 63/433,863, filed Dec. 20, 2022, which is incorporated by reference herein in its entirety.

### BACKGROUND

Data visualization is an essential component of genomic data analysis. Next-generation sequencing (NGS) and array-based profiling methods generate large quantities of diverse types of genomic data and are enabling researchers to study the genome at unprecedented resolution. Although much of the analysis can be automated, human interpretation and judgment, supported by rapid and intuitive visualization, is essential for gaining insight and elucidating complex biological relationships. Genome browsers are applications (e.g., browser applications) that display sequencing data. Genome browsers may be web-based browsers for displaying sequencing data. Genome browsers display alignments, variants, and/or other types of genomic annotations from multiple samples for performing complex variant analysis. Although genome browsers are often used to view genomic data from public sources, genome browsers may also support researchers who wish to visualize and explore their own data sets or those from colleagues. To that end, genome browsers support flexible loading of local and remote data sets, and are optimized to provide high-performance data visualization and exploration on standard desktop systems.

Fetching data from entire genomic files, while at the whole-genome view, or even relatively large portions of the genome, produces amounts of data that are unsupported by genome browsers. This may result in genome browsers being unable to display certain levels of information stored in genomic files when a user selects a certain amount of information to be displayed.

### SUMMARY

Systems, methods, and apparatus are described herein for aggregating genome data into bins with summary data from non-summary files (e.g., BED files, FASTA files, BAM files, etc.) at various levels. By summarizing and/or aggregating data from non-summary files, bite-size pieces of data from genomic files may be accessed and/or displayed at various levels of resolution. A computing device may be configured to receive genome data associated with a genome. The genome data may be received in an alignment map file. The alignment map file may be a binary alignment map (BAM) file, a sequence alignment map (SAM) file, and/or another non-summary file. The computing device may be configured to generate an aggregate file using the received genome data. The aggregate file may comprise a plurality of bins at a plurality of depths (e.g., levels). The plurality of bins may comprise a first set of bins at a first depth, a second set of bins at a second depth, and a third set of bins at a third depth. A bin of the first set of bins may comprise a plurality of bins of the second set of bins at the second depth. A bin of the second set of bins may comprise a plurality of bins of the third set of bins at the third depth. Each of the plurality of bins may occupy an equal sized space of memory.

The aggregate file may comprise a header that indicates a name length, a genome name, a reference length, and/or a scale factor. The scale factor may indicate how many bins of a proximate depth are comprised within a respective one of the plurality of bins. For example, the scale factor may indicate how many bins of a lower depth are combined into a respective one of the plurality of bins at a next higher depth. Additionally or alternatively, the scale factor may indicate how many bins of the second set of bins are comprised within the third set of bins and how many bins of the first set of bins are comprised within the second set of bins. The name length and the genome name may identify the genome. The computing device may be configured to determine a minimum depth and a maximum depth for the aggregate file based on the reference length and the scale factor.

The computing device may be configured to determine summary data for respective reads, variants, and/or annotated regions associated with respective portions of the genome covered by respective bins of the plurality of bins. The summary data may be determined based on the received genome data and/or the aggregate file. The summary data may comprise an average quality, an average depth, and/or one or more nucleotide proportions. The computing device may be configured to read the BAM file to identify the respective reads for a respective bin, for example, when determining the summary data for the respective bin.

The computing device may be configured to store the summary data for the respective reads, variants, and/or annotated regions in the respective bins of the plurality of bins that cover the respective portions of the genome associated with the respective reads, variants, and/or annotated regions. A read that overlaps two of the plurality of bins may be assigned to one of the two bins based on how much it overlaps each of the two bins. The second set of bins may comprise summary data associated with a plurality of the first set of bins at the first depth. The third set of bins may comprise summary data associated with a plurality of the second set of bins at the second depth. Each of the bins at a specific depth may comprise summary data of an equal portion of the genome.

The computing device may be configured to display a portion of the summary data in response to a selection of a genomic region by a user. The displayed portion of summary data may be associated with one or more of the bins of the plurality of bins that correspond with the genomic region selected by the user. The displayed portion of summary data may correspond with a depth of the plurality of depths. The computing device may be configured to determine the depth for the displayed portion of summary data based on the genomic region selected by the user. The computing device may be configured to identify one or more bins at the determined depth that overlap the genomic region selected by the user.

The portion of summary data may be displayed using one or more display conditions, for example, to represent relative differences in the summary data between the one or more bins of the displayed portion of summary data. The one or more display conditions comprise color, opacity, and/or height. The computing device may be configured to identify a location in the aggregate file that corresponds to the genomic region selected by the user. The location in the aggregate file may comprise a specific bin of the plurality of bins at a specific depth of the plurality of depths.

### BRIEF DESCRIPTION OF THE DRAWINGS

FIG. 1A illustrates a schematic diagram of a system environment.

FIG. 1B illustrates an example of one or more sequencing subsystems that may be implemented for identifying variants.

FIG. 2 is a block diagram of an example computing device.

FIG. 3A is a diagram depicting an example layout for an aggregate file.

FIG. 3B is a diagram depicting an alternate example bin format for the aggregate file shown in FIG. 3A.

FIG. 4A is an illustration depicting an example aggregate viewer for displaying summary data associated with genome data.

FIG. 4B is a partial detailed view of the example aggregate viewer shown in FIG. 4A.

FIG. 5 is a flowchart depicting an example process for generating an aggregate file and displaying a portion of summary data stored in the aggregate file.

FIG. 6A is a diagram depicting an example format of an index file.

FIG. 6B is a diagram depicting an example format of an aggregate file for use with the index file of FIG. 6A.

FIG. 7 an illustration depicting another example aggregate viewer for displaying summary data associated with genome data.

FIG. 8 is a flowchart depicting an example process for generating an aggregate file and an index file for displaying data associated with a selected genomic region.

### DETAILED DESCRIPTION

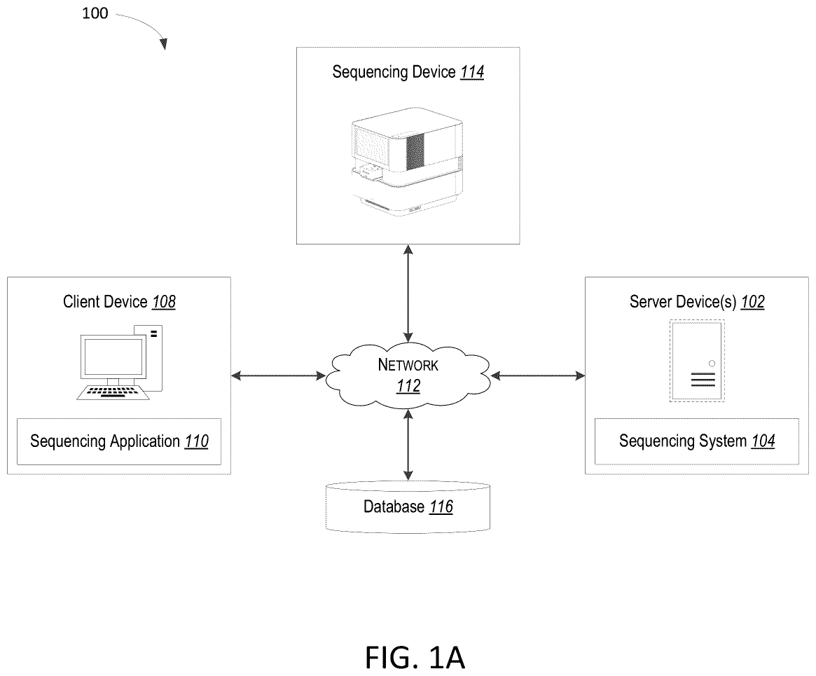

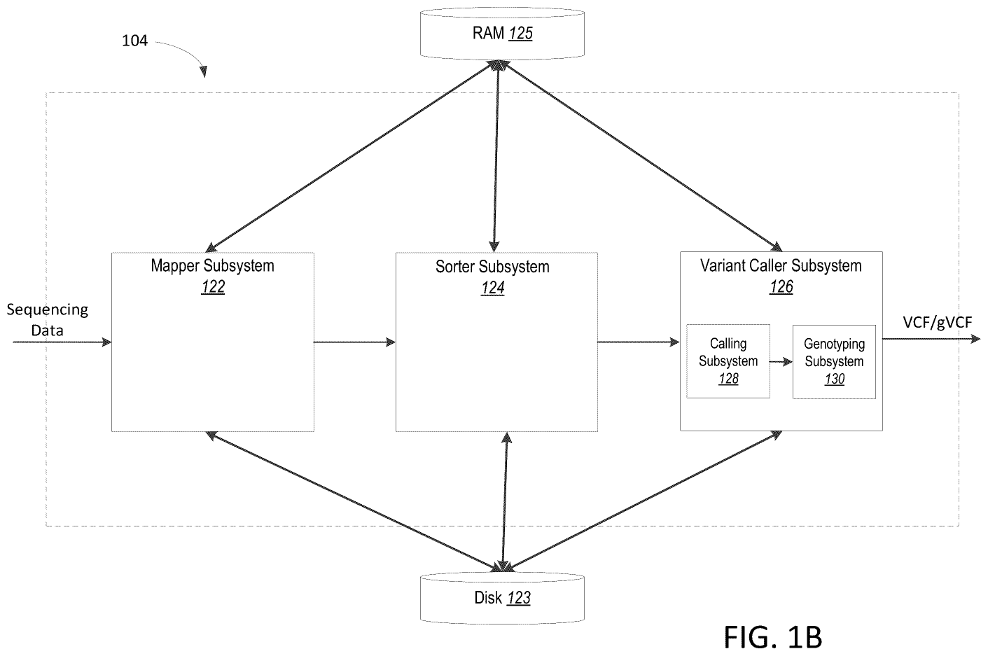

FIG. 1A illustrates a schematic diagram of a system environment (or “environment”) 100, as described herein. As illustrated, the environment 100 includes one or more server device(s) 102 connected to a client device 108 and a sequencing device 114 via a network 112.

As shown in FIG. 1A, the server device(s) 102, the client device 108, and the sequencing device 114 may communicate with each other via the network 112. The network 112 may comprise any suitable network over which computing devices can communicate. The network 112 may include a wired and/or wireless communication network. Example wireless communication networks may be comprised of one or more types of radio frequency (RF) communication signals using one or more wireless communication protocols, such as a cellular communication protocol, a wireless local area network (WLAN) or WIFI communication protocol, and/or another wireless communication protocol. In addition, or in the alternative to communicating across the network 112, the server device(s) 102, the client device 108, and/or the sequencing device 114 may bypass the network 112 and may communicate directly with one another.

As indicated by FIG. 1A, the sequencing device 114 may comprise a device for sequencing a biological sample. The biological sample may include human and non-human deoxyribonucleic acid (DNA) to determine individual nucleotide bases of nucleic-acid sequences (e.g., sequencing by synthesis). The biological sample may include human and non-human ribonucleic acid (RNA). The sequencing device 114 may analyze nucleic-acid segments and/or oligonucleotides extracted from samples to generate nucleotide reads and/or other data utilizing computer implemented methods and systems described herein either directly or indirectly on the sequencing device 114. More particularly, the sequencing device 114 may receive and analyze, within nucleotide-sample slides (e.g., flow cells), nucleic-acid sequences extracted from samples. The sequencing device 114 may utilize SBS to sequence nucleic-acid segments into nucleotide reads.

As further indicated by FIG. 1A, the server device(s) 102 may generate, receive, analyze, store, and/or transmit digital data, such as data for determining nucleotide-base calls or sequencing nucleic-acid polymers. As shown in FIG. 1A, the sequencing device 114 may generate and send (and the server device(s) 102 may receive) nucleotide reads and/or other data for being analyzed by the server device(s) 102 for base calling and variant calling. The server device(s) 102 may also communicate with the client device 108. In particular, the server device(s) 102 may send data to the client device 108, including sequencing data or other information and the server device(s) 102 may receive input from the user via client device 108.

The server device(s) 102 may comprise a distributed collection of servers where the server device(s) 102 include a number of server devices distributed across the network 112 and located in the same or different physical locations. Further, the server device(s) 102 may comprise a content server, an application server, a communication server, a web-hosting server, or another type of server.

As further shown in FIG. 1A, the server device(s) 102 may include a sequencing system 104. The sequencing system 104 may analyze nucleotide reads and/or other data, such as sequencing metrics received from the sequencing device 114, to determine nucleotide base sequences for nucleic-acid polymers. For example, the sequencing system 104 may receive raw data from the sequencing device 114 and may determine a nucleotide base sequence for a nucleic-acid segment. The raw data may be received from the sequencing device 114 in a file format, such as a FASTA or a FASTQ file, that is capable of being recognized for processing. A FASTA and a FASTQ file may each include a text file that contains the sequence data from clusters that pass filter on a flow cell. The FASTA and the FASTQ format are each a text-based format for storing both a biological sequence (e.g., such as a nucleotide sequence). The FASTA may include the nucleotide sequence data. The FASTQ may store the nucleotide sequence data and its corresponding quality scores. The sequencing system 104 may process the sequencing data to determine the sequences of nucleotide bases in DNA and/or RNA segments or oligonucleotides.

In addition to processing and determining sequences for biological samples, the sequencing system 104 may generate a file for processing and/or transmitting to other devices. The files that are generated may be in a sequence alignment/map (SAM) format (e.g., a SAM file), a binary alignment/map (BAM) format (e.g., a BAM file), a compressed reference-oriented alignment map (CRAM) format (e.g., a CRAM file), and/or another file format for processing and/or transmitting to other devices. The SAM format may be an alignment format for storing reads aligned to a reference genome. The SAM may store biological sequences aligned to a reference sequence. The SAM format may support short and long reads (e.g., up to 128 Mb) produced by different sequencing devices 114. The SAM format may be a text format file that is human-readable. Though a conversion may be made of data in a FASTA file straight to a BAM file. The SAM file may include a header section and an alignment section that includes alignment information data for aligning one or more reads of the sequencing data generated by the sequencing device 114 with a reference sequence. The header section may include a reference sequence dictionary (e.g., referred to as SQ), a reference sequence name (e.g., referred to as SN) for the reference sequence chromosome in the dictionary, and/or a reference sequence length (e.g., referred to as LN). The alignment information data may include a query template name (e.g., referred to as QNAME), a flag that indicates how the sequencing data is mapped onto a reference sequence, a reference sequence name (e.g., referred to as RNAME), a position at which a read sequence starts on the reference sequence, a mapping quality (e.g., referred to as MAPQ), a CIGAR string that indicates matches and/or differences (e.g., insertions, deletions, or other modifications) between the read and the reference sequence, a reference name of a mate or next read (e.g., referred to as RNEXT), a position of the mate or next read (e.g., referred to as PNEXT), a template length (e.g., referred to as TLEN), a sequence that provides information on the exact sequence (e.g., referred to as SEQ), and/or quality (e.g., referred to as QUAL) that indicates the base quality of the read. The mapping quality, or MAPQ, score may indicate how well the read maps to the reference genome. The mapping quality score may be rounded to a nearest integer. The read alignment is the process of figuring out where in the genome a sequence is from. Once the alignment is performed, the mapping quality or the mapping quality score (e.g., MAPQ) of a given read quantifies the probability that its position on the genome is correct. The mapping quality is encoded in the phred scale where P is the probability that the alignment is not correct. The mapping quality is associated with several alignment factors, such as the base quality of the read, the complexity of the reference genome, and paired-end information. The MAPQ value can be used as a quality control of the alignment results. The proportion of reads aligned with an MAPQ higher than 20 is often used for downstream analysis. The BAM format may maintain the same information in a SAM file, but in a compressed, binary format that is machine-readable. BAM files may show the alignments of the reads received in the sequencing data from the sequencing device 114, as described with regard to the SAM file, but in a binary format. CRAM files may be stored in a compressed columnar file format for storing biological sequences.

The client device 108 may generate, store, receive, and/or send digital data. In particular, the client device 108 may receive sequencing metrics from the sequencing device 114. Furthermore, the client device 108 may communicate with the server device(s) 102 to receive one or more files comprising nucleotide base calls and/or other metrics. The client device 108 may present or display information pertaining to the nucleotide-base call within a graphical user interface to a user associated with the client device 108.

The client device 108 illustrated in FIG. 1A may comprise various types of client devices. In examples, the client device 108 may include non-mobile devices, such as desktop computers or servers, or other types of client devices. In other examples, the client device 108 may include mobile devices, such as laptops, tablets, mobile telephones, or smartphones.

As further illustrated in FIG. 1A, the client device 108 may include a sequencing application 110. The sequencing application 110 may be a web application or a native application stored and executed on the client device 108 (e.g., a mobile application, desktop application). The sequencing application 110 may include a genome viewer or a genome browser for displaying information on the client device 108. The sequencing application 110 may include instructions that (when executed) cause the client device 108 to receive data from the sequencing device 114 and/or server device 102 and present, for display at the client device 108, data to the user of the client device 108, such as data from a variant call file.

As further illustrated in FIG. 1A, the environment 100 may include a database 116. The database 116 can store information such as variant call files, sample nucleotide sequences, nucleotide reads, nucleotide-base calls, sequencing metrics, population data, and/or other data as described herein. The server device(s) 102, the client device 108, and/or the sequencing device 114 may communicate with the database 116 (e.g., via the network 112) to store and/or access information, such as variant call files, sample nucleotide sequences, nucleotide reads, nucleotide-base calls, sequencing metrics, population data, and/or other data as described herein.

The environment 100 may be included in a local network or local high-performance computing (HPC) system. The environment 100 may be included in a cloud computing environment comprising a plurality of server devices, such as server device(s) 102, having software and/or data distributed thereon. The sequencing system 104 may be implemented to operate one or more subsystems as described herein, and may be distributed across server devices 102 having access to the database 116 via the network 112 in a cloud-based computing system.

Though FIG. 1A illustrates the components of environment 100 communicating via the network 112, it will be appreciated that the components of environment 100 may communicate directly with each other, for example, bypassing the network 112. For example, the client device 108 may communicate directly with the sequencing device 114.

The sequencing system 104 may comprise one or more sequencing subsystems used to analyze the sequencing data received from the sequencing device 114 and/or identify variants in the sequencing data. The nucleotide-base call may indicate a determination or prediction of the type of nucleotide base that has been incorporated within an oligonucleotide on a nucleotide-sample slide (e.g., read-based nucleotide-base calls) or a determination or prediction of the type of nucleotide base that is present at a genomic coordinate or genomic region within a sample genome. For example, a nucleotide-base call may include a base call corresponding to a genomic coordinate and a reference genome, such as an indication of a variant or a non-variant at a particular location corresponding to the reference genome. A nucleotide-base call may refer to the base that is detected at a position in a read together with a quality score that indicates a confidence in that call. The base call may allow for detection of a mutation or variant based on a comparison between the base call in each read that spans a position and the base that is presented in the reference genome at the same position. The variant may include, but is not limited to, a single nucleotide polymorphism (SNP), an insertion or a deletion (indel), or a base call that is part of a structural variant. An insertion changes the DNA sequence by adding one or more nucleotides to the sequence as compared to the reference genome. A deletion changes the DNA sequence by removing at least one nucleotide from the sequence as compared to the reference genome. The deleted DNA may alter the function of the affected protein or proteins. A single nucleotide-base call can comprise an adenine call, a cytosine call, a guanine call, or a thymine call for DNA (abbreviated as A, C, G, T) or a uracil call (instead of a thymine call) for RNA (abbreviated as U). A mutation may include a single change or difference in the genetic sequence. The variant may comprise a sequence that comprises one or more mutations.

FIG. 1B shows an example of one or more sequencing subsystems that may be implemented by the sequencing system 104 for identifying variants. As shown in FIG. 1B, the sequencing system 104 may implement a mapper subsystem 122, a sorter subsystem 124, and/or a variant caller subsystem 126. The mapper subsystem 122 may be implemented to align the reads in sequencing data received from the sequencing device 114 and/or stored at the server device(s) 102. The reads in the sequencing data produced by the sequencing device 114 and/or generated and stored in the files by the server device(s) 102 may not be included in a single sequence with all DNA information. Instead, the sequencing data produced by the sequencing device 114 and/or generated in the files by the server device(s) 102 may include a number of short subsequences, or reads, with partial DNA information. Read alignment may be performed by the mapper subsystem 122 to map reads to a reference genome and identify the location of each individual read on the reference genome. The mapper subsystem 122 may stream unaligned reads from the sequencing data as FASTQ or ILLUMINA individual base call (BCL) files and perform read alignment on the sequencing data therein. FASTQ files can contain up to millions of entries and can be several megabytes (Mbs) or gigabytes (GBs) in size. The mapper subsystem 122 may output the aligned reads in an aligned BAM file, as described herein.

The BAM files may include a header section and an alignment section. The header section may include information about the file, such as sample name, sample length, and alignment method. The alignment section may include a read name, read sequence, read quality, alignment information, and other custom tags for the read. For each read or read pair, the alignments section may include a read group. The read group may include a subset of reads on a flow cell from the same lane, sample, and/or library prep. Different read groups may have different coverage or different depth. The depth may be determined by a number of reads aligned to a location in the sequence with a certain quality. The depth may be determined by a number of reads aligned to a location in the sequence with a certain quality. The number of reads may be determined for one or more read groups. The alignment section may include a barcode tag that indicates a demultiplexed sample identifier associated with the read. The alignment section may include a single-end alignment quality. The alignment section may include an edit distance tag, which records the Levenshtein distance between the read and the reference.

Read alignment may be performed using a hash table. A hash table may be built for the genome reference, which may enable a sub-portion of the read, or seed, to be mapped to the genome. The location of the read may be determined from the result of seed extension at each of its mapping locations. The mapper subsystem 122 may use a hash table index of a reference genome to map many overlapping seeds from each read to exact matches in the reference. The hash table may be constructed from any chosen reference with a multi-threaded tool, and loaded into random access memory (RAM) 116. For example, the RAM 116 may comprise a field programmable gate array (FPGA)-board dynamic RAM (DRAM) on the server device(s) 102. The hash table may be stored on the RAM 116 prior to mapping operations performed by the mapper subsystem 122. The read-mapping process may be performed by FPGA logic on the RAM 116.

After the read alignment is performed at the mapper subsystem 122, the aligned sequencing data may be passed downstream to the sorting subsystem 124 to sort the reads by reference position, and polymerase chain reaction (PCR) or optical duplicates are optionally flagged. An initial sorting phase may be performed by the sorter subsystem 124 on aligned reads returning from the RAM 125. Final sorting and duplicate marking may commence when mapping completes. The sorter subsystem 124 may write another BAM file that includes sorted sequencing data to RAM 125 for being accessed downstream by the variant caller subsystem 126.

The variant caller subsystem 126 may be used to call variants from the aligned and sorted reads in the sequencing data. For example, the variant caller subsystem may receive the sorted BAM file as input and process the reads to generate variant data to be included in a variant call file (VCF) or a genomic variant call format (gVCF) file as output from the variant caller subsystem 126.

The variant caller subsystem 126 may comprise a calling subsystem 128 and/or a genotyping subsystem 130. As the variant caller subsystem 126 receives the sequencing data, the calling subsystem 128 may identify callable regions with sufficient aligned coverage. The callable regions may be identified based on a read depth. The read depth may represent a number of times a particular base is represented within each of the reads in the sequencing data. Sometimes the wrong base may be incorporated into a DNA fragment identified in the sequencing data. For example, a camera in the sequencing device 114 may pick up the wrong signal, the mapper subsystem 122 may misplace a read, or a sample may be contaminated to cause an incorrect base to be called in the sequencing data. By sequencing each fragment numerous times to produce multiple reads, there is a confidence or likelihood that identified variants are true variants and not artefacts from the sequencing process. The read depth represents the number of times each individual base has been sequenced or the number of reads in which the individual base appears in the sequencing data. The higher the read depth, the greater the level of confidence in variant calling.

The callable regions may be the regions that are passed downstream to the genotyping subsystem 130 for calling variants from the callable region. For example, the genotyping subsystem 130 may compare the callable region to a reference genome for variant calling. The calling subsystem 128 may identify a callable region when the read depth of the sequencing data is above a callable region depth threshold. For example, the calling subsystem 128 may identify a callable region in the sequencing data when the read depth of one or more sequence fragments is above a depth threshold of one. After the callable region is identified, the calling subsystem 128 may pass the callable region to the genotyping subsystem 130, which may turn the callable region into an active region for generating potential positions in the active region where there may be variants. The genotyping subsystem 130 may identify a probability or call score of whether a potential position includes a variant.

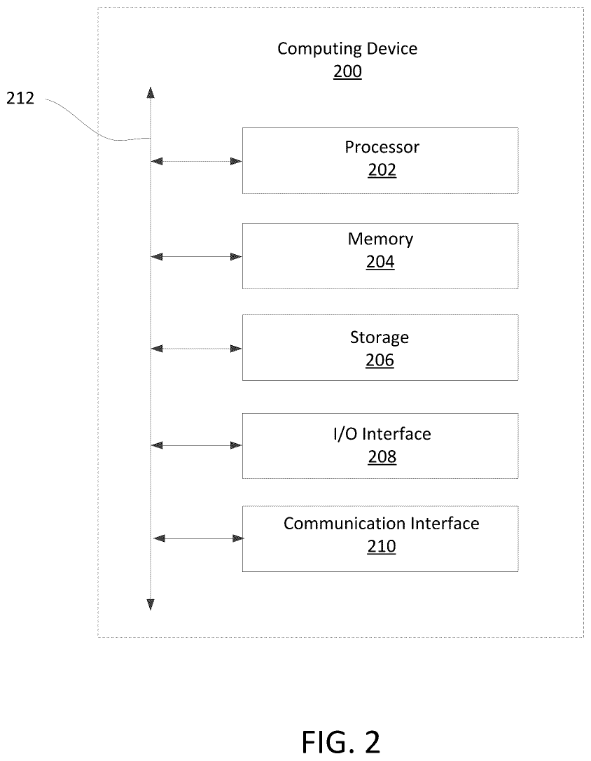

FIG. 2 is a block diagram illustrating an example computing device 200. One or more computing devices such as the computing device 200 may implement one or more features for aggregating genome data into bins with summary data at various levels and displaying the summary data. For example, the computing device 200 may comprise one or more of the client device 108, the sequencing device 114, and/or the server device 102 shown in FIG. 1A. As shown by FIG. 2, the computing device 200 may comprise a processor 202, a memory 204, a storage device 206, an I/O interface 208, and a communication interface 210, which may be communicatively coupled by way of a communication infrastructure 212. It should be appreciated that the computing device 200 may include fewer or more components than those shown in FIG. 2.

The processor 202 may include hardware for executing instructions, such as those making up a computer program. The instructions may be computer-executable instructions retrieved from the memory 204 for configuring the processor 202, as described herein. In examples, to execute instructions for dynamically modifying workflows, the processor 202 may retrieve (or fetch) the instructions from an internal register, an internal cache, the memory 204, or the storage device 206 and decode and execute the instructions. The memory 204 may be a volatile or non-volatile memory used for storing data, metadata, computer-readable or machine-readable instructions, and/or programs for execution by the processor(s) for operating as described herein. The storage device 206 may include storage, such as a hard disk, flash disk drive, or other digital storage device, for storing data or instructions for performing the methods described herein.

The I/O interface 208 may allow a user to provide input to, receive output from, and/or otherwise transfer data to and receive data from the computing device 200. The I/O interface 208 may include a mouse, a keypad or a keyboard, a touch screen, a camera, an optical scanner, network interface, modem, other known I/O devices or a combination of such I/O interfaces. The I/O interface 208 may include one or more devices for presenting output to a user, including, but not limited to, a graphics engine, a display (e.g., a display screen), one or more output drivers (e.g., display drivers), one or more audio speakers, and one or more audio drivers. The I/O interface 208 may be configured to provide graphical data to a display for presentation to a user. The graphical data may be representative of one or more graphical user interfaces and/or any other graphical content.

The communication interface 210 may include hardware, software, or both. In any event, the communication interface 210 may provide one or more interfaces for communication (such as, for example, packet-based communication) between the computing device 200 and one or more other computing devices or networks. The communication may be a wired or wireless communication. As an example, and not by way of limitation, the communication interface 210 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 210 may facilitate communications with various types of wired or wireless networks. The communication interface 210 may also facilitate communications using various communication protocols. The communication infrastructure 212 may also include hardware, software, or both that couples components of the computing device 200 to each other. For example, the communication interface 210 may use one or more networks and/or protocols to enable a plurality of computing devices connected by a particular infrastructure to communicate with each other to perform one or more aspects of the processes described herein. To illustrate, the sequencing process may allow a plurality of devices (e.g., a client device, sequencing device, and server device(s)) to exchange information such as sequencing data and error notifications.

The computing devices described herein may be implemented to display information to a user. For example, the information may be displayed using a local application, such as a genome viewer, that displays information stored locally on the computing device and/or retrieved from a remote computing device (e.g., via a network). Genome viewers may include a genome browser, which may be referred to as an Integrative Genomics Viewer (IGV), or other applications (e.g., browser applications or other command line applications) that display sequencing data. Genome viewers may be web-based browsers for displaying the sequencing data. For example, a genome viewer may be executed as a local application (e.g., sequencing application 110 shown in FIG. 1A or portion thereof) operating on a client device to display information generated at one or more remote computing devices. In the example shown system shown in FIG. 1A, the client device 108 may execute a genome viewer to retrieve and display sequencing data from one or more server devices 102 in response to user input.

The sequencing data to be displayed in the genome viewer may be stored in one or more files. For example, the sequencing data may be stored in a browser extensible data (BED) format or a BedGraph format. The BED file format may be a text file format used to store genomic regions as coordinates and associated annotations. The data in the BED file format may be presented in the form of columns separated by spaces or tabs, where each row may represent a region of the genome and associated annotations or values. The BED file may include three or more columns that indicate the sections or regions of the chromosome and/or other information related to the sections or regions of the chromosome. For example, the BED file may include a chromosome number in a first column, a start position of the section or region of the chromosome in a second column, and a stop position of the section or region in a third column. The start and stop positions may indicate the coordinates of the section or region in the genome. Provided below is an example illustrating the first three rows or lines of a BED file.

| Chr | Start | Stop |
|---|---|---|
| chr3 | 0 | 173 |
| chr3 | 173 | 52703 |
| chr3 | 52703 | 105233 |
| . . . | | |

The BED file may include additional columns that include other information about the identified sections or regions. The BED file may include many rows that each indicate the sections or regions of the chromosome and related information. A BedGraph file may also store coordinate information for sections or regions in the genome, but may be used to show coverage depth of sequencing over a genome. The BedGraph file is based on a BED file and includes similar data, such as a chromosome number, a start position, and/or a stop position, as described herein. The BedGraph file may also include a column that includes score data for the sections or regions in the genome. The score data may also be included in the BED file, but in a different column (e.g., column 4 in BedGraph file and column 5 in BED file). The score (e.g., BED score) may be a value between 0 and 1000 (e.g., though other values, such as p-values or mean enrichment values may be used) to indicate regions of statistically significant signal enrichment. The score associated with each enriched interval may be identified as the mean signal value across the interval.

The sequencing data may be stored in VCF for gVCF format that includes information on variants in the sequencing data. The VCF or gVCF file may be a digital file generated in a publicly available standard text format that includes a number of predefined fields of summary information related to a sample, such as genotype variant data, related to the sample to which the VCF or gVCF file corresponds. The summary information in the VCF or gVCF file may include genotype variant data about the variants and non-variant genomic blocks at specific genomic coordinates, including meta-information lines, a header line, and data lines where each data line contains information about a single nucleotide-base call (e.g., a single variant). The genotype variant data may include one or more nucleotide-base calls (e.g., variant calls) along with other information pertaining to the nucleotide-base calls (e.g., variant calls, quality, mapping alignment, and other metrics). To provide an example of the size of the standard VCF or gVCF files that are used for sequencing analysis, we describe herein the plurality of fields that are utilized in the standard format. For example, the plurality of fields in the VCF or gVCF file(s) may include a genotype (GT) field, a genotype quality (GQ) field, a minimum of genotype quality (GQX) field, a filtered base call depth (DP) field, a base calls filtered from input (DPF) field, an allelic depth (AD) field, a read depth associated with indel (DPI) field, a mapping qualities (MQ) field, a filter (FT) field, a quality (QL) field, a phred-scaled genotype likelihood (PL) field, and a reference allele, one or more alternate alleles+genotype (GT) field, a contig name (CHROM), the start and end position of the record (POS, END), the reference allele sequence (REF), and/or the sequence of one or more alternate alleles (ALT). VCF files be multi-sample files and/or include fields (e.g., GT and/or AD fields) for more than one sample. VCF files may include variant calls for many types of variants, include single nucleotide, multi-nucleotide, indel, copy number variants, structural variants, and/or short tandem repeat variants.

Genome browsers may display alignments and variants from multiple samples for performing complex variant analysis. Although genome browsers are often used to view genomic data from public sources, genome browsers may also support researchers who wish to visualize and explore their own data sets or those from colleagues. To that end, genome browsers support flexible loading of local and remote data sets, and are optimized to provide high-performance data visualization and exploration on standard desktop systems.

In order to display the alignments and variants for performing variant analysis, one or more computing devices may retrieve and process the data from multiple files stored in memory. In the example system shown in FIG. 1A, the one or more server devices 102 may access sequencing data stored in a FASTA or FASTQ file, a BED file or BedGraph file, VCF or gVCF file, and/or data stored in a BAM file, and provide the data to the genome browser executing on the client device 108 (e.g., via the sequencing application 110) in response to a request for the sequencing data. For example, a user may request sequencing data related to one or more select genomic regions, or an entire genome, via the genome browser executing on the client device 108 and the request may be sent to the one or more server devices 102. In an example, the request may include a chromosome coordinate range, which may be represented as chr3:235595-335695, or an indication of a chromosome coordinate range for which information is to be displayed in the genome browser. For example, the chromosome range may be automatically determined to be a predefined position (e.g., left and/or right) or range around a user selection of a genomic region in the genome browser. The predefined position (e.g., left and/or right) or range may be larger when the user is zoomed out to view a larger genomic region and smaller when the user is zoomed in to view a smaller genomic region. The user may view different sequencing data for positions or genomic regions (e.g., of the same predefined size) at the same zoom level by scrolling through the positions or genomic regions and retrieving updated sequencing data for the updated positions or genomic regions, or the user may zoom in or out to view sequencing data of genomic regions of different sizes.

The one or more server devices 102 may access the data stored in the FASTA or FASTQ file, BED or BedGraph file, VCF or gVCF file, and/or a BAM file, and provide the requested data to the genome browser on the client device for display. When processing the data in the BAM file, indexing methods may be employed to expedite the processing of information in the BAM file. For example, when processing the data in the BAM file, a BAM index file may also be referenced from memory. As the BAM file may store large amounts of aligned sequencing data, the BAM index file may operate as a lookup to allow the one or more server devices 102 (e.g., the sequencing system 104 operating thereon) to jump directly to specifically indexed portions of the BAM file to access requested information without reading through all of the sequencing data stored in the BAM file (e.g., other several hundred GB of data in BAM file) prior to the needed portions. The BAM index file may allow for the retrieval of alignments in the sequencing data that overlap a specific location without having to read all of the prior data. The BAM index file may identify the chromosome and position at which the BAM file may be read to obtain related information.

Genome browsers may have trouble displaying sequencing data at various zoom levels, each of which may provide different levels of detail for one or more portions of the genomic regions being displayed. For example, a user of a genome browser may select a desired zoom level and/or a desired portion of a genome for being displayed. The genome browser may attempt to display the relevant data for a portion of the genome that is selected. If the genome browser is zoomed out too far (e.g., beyond a zoom threshold), there may be too much data to display in the genome browser and the data may not be viewable. The FASTA and FASTQ files may be hundreds of megabytes (MBs) to 3 gigabytes (GBs). FASTA files for a whole genome sequencing run may be 30-200 GB. The BED files may be hundreds of kilobytes (KBs) (e.g., 500 to 900 KBs) or MBs (e.g., 1 to 5 MBs) in size. The BedGraph may be several hundred MBs (e.g., 500 to 900 MBs) or gigabytes (GBs) (e.g., 1 to 5 GBs) in size. The BAM files may be between 50 GBs to over 200 GBs in the compressed format and/or 100 GBs to 500 GBs decompressed. BAM files may have a compression ratio of around 4:1, such that a SAM file of 8 GB may compress to 2 GB in a BAM format. After conversion to human-readable form, the size of the BAM may be multiplied from 1.5 to 10 times the compressed file size. For example, a 200 GB BAM, once decompressed and decoded to something readable, could be over a Terabyte of data. Decompressed VCFs and gVCFs may be hundreds of GBs decompressed. The amount of data that is being requested may not be supported by the genome browser itself. For example, the genome browser may be limited to displaying a certain amount of data (e.g., hundreds of MBs or up to 200 or 300 GBs of data for a web-based genome browser) at a maximum. The genome browser may operate using the RAM on a system and may not have access to a system hard driver for storing data. The genome browser code itself may use up to a GB or over a GB of RAM and may have to share the RAM with other applications running on the system. Referring again to the example system shown in FIG. 1A, the amount of data to be provided in response to the request from the user may take a relatively large amount of time to transmit over the network 112 from the one or more server devices 102 to the client device 108. For example, the processing and/or communication of the sequencing data in the BED/BedGraph files and/or the BAM files may take tens of minutes to over an hour, as well as occupying dedicated network resources for that time period, depending on the type of network 112.

When there is too much data to display (e.g., data above a threshold) and/or the genome browser is zoomed out beyond the zoom threshold, the genome browser may prompt the user to zoom in to view the data. Stated differently, the desired zoom level and/or desired portion of the genome may correspond to a large area of the genome and the genome browser may be unable to be display all of the data for the large area. In addition, when genome browsers attempt to display large amounts of data (e.g., that occupy memory and/or processing resources above a threshold level allocated to the genome browser), the genome browsers may become slow and/or unresponsive. Genome browsers attempting to display large amounts of data (e.g., that occupy memory and/or processing resources above a threshold level allocated to the genome browser) may slow computing performance and consume larger amounts of power.

Indexing methods may be used to assist in retrieving data related to portions of sequencing data in a genome region and faster processing of requests from the genome browsers. However, the indexing methods retrieve all of the data in the BAM file for the genomic region. For larger genome regions (e.g., having over a GB of data in the BAM file), computing devices may be unable to obtain and/or process the requested data and display the requested data in the genome browser in a manner that is responsive to the input received from the user (e.g., responsive to user input to zoom in/out of regions of the genome).

The indexing methods used by conventional genome browsers were also not developed for visualization, but rather for rapid retrieval of sections of large files. In order to view data across a large area (e.g., such as the whole genome), users typically produce a subset of the data, then use the same indexing tools on the subset of data. This only handles a single zoom level. To view data at multiple zoom levels, multiple files for various subsets of data may be generated. Each of the multiple files may then be indexed and the genome browser could look at different files depending on the zoom level.

An intermediate file (e.g., aggregate file) may be created from received genome data. The intermediate file may separate the received genome data into equal sized portions at various levels (e.g., zoom levels). The intermediate file may summarize the genome data associated with each of the portions to enable visualization of relevant data for those portions of the genome. The intermediate file may summarize genome data for display at respective zoom levels. For example, summary data may be displayed in a genome viewer instead of the genome data stored in a non-summary file (e.g., BED files, FASTA files, BAM files, etc.) or original file in which full genome data may be stored. The summary data may be generated from non-summary files (e.g., BED files, FASTA files, BAM files, etc.) at various levels. By summarizing and/or aggregating data from non-summary files, bite-size pieces of data from genomic files may be accessed and/or displayed at various levels of resolution without the same demand on memory and/or networking resources. In examples, the summary data from the intermediate file may be displayed when the original genome data is too large to display. The intermediate file (e.g., aggregate file) may be preprocessed and stored with summary data from the FASTA file, the BED file, the BedGraph file, the BAM file, and/or VCF/gVCF files in bins for direct access of the summary data in response to requests to display different levels of sequencing data for portions of a genome. The intermediate file (e.g., aggregate file) may store smaller amounts of sequencing data, such that even if a request is received for displaying sequencing data for the whole genome the data will be provided responsive to the user inputs (e.g., input to zoom in/out of regions of the genome). The summary data may be limited to a predefined number of parameters to limit the amount of data being retrieved/processed. For example, the summary data may include a chromosome identifier, a position, a MAPQ, and/or a string of a read. Limiting the number of parameters being provided in the summary data may reduce the memory utilized to process a request for displaying the sequencing data in a genome viewer. For example, the amount of data used to retrieve the summary data in response to a request may be less than five times the memory that may be used to read a BAM file to process the same request, even when indexing methods are implemented. As illustrated in the example system of FIG. 1A, the one or more server devices 102 may be responsive to the requests to provide the sequencing data to the client device 108 over the network 112 using the intermediate file and the summary data stored therein.

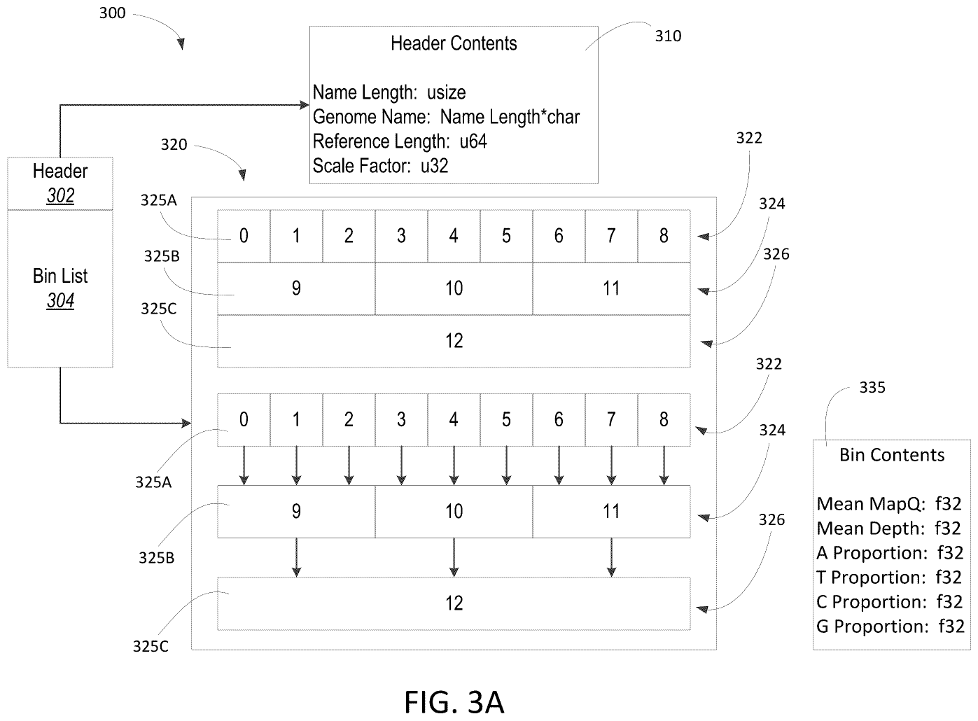

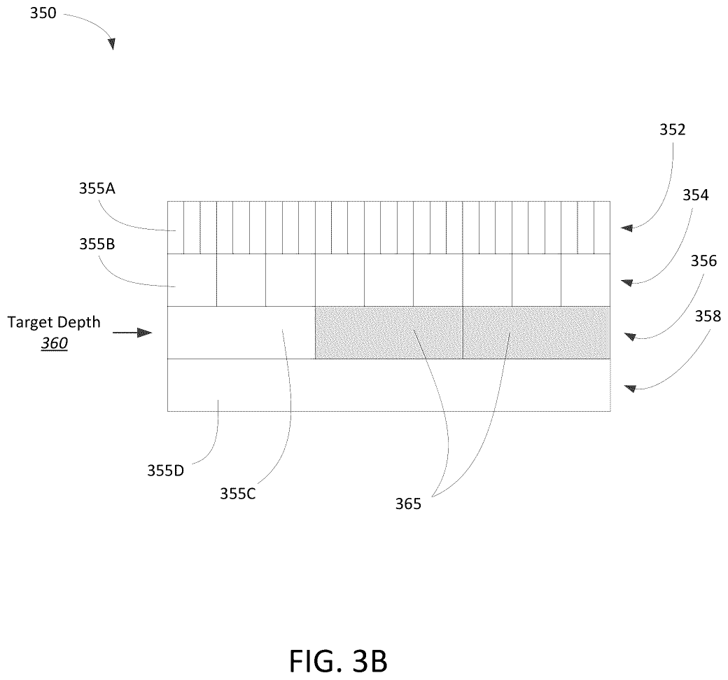

FIG. 3A depicts an example layout for an aggregate file 300, which may be an example of a summary file that includes summary data. The aggregate file 300 may enable viewing data (e.g., summary data) associated with an entire genome. For example, genome data may be received from sequencing of the genome. The aggregate file 300 may enable viewing data associated with different zoom levels for the genome. The aggregate file 300 may save processing resources by eliminating the need for a separate index file to search the aggregate file 300 and/or by reducing the amount of data displayed for a selected portion of the genome. The aggregate file 300 may be used to store summary data associated with genome data (e.g., genome sequencing data). For example, the aggregate file 300 may be used to store summary data from a SAM file or a BAM file. The aggregate file 300 may be generated by a computing device (e.g., such as the client device 108, the server device 102, and/or the computing device 200 shown in FIGS. 1A and 2, respectively).

The aggregate file 300 may comprise a header 302 and/or a bin list 304. The bin list 304 may be a list of a plurality of bins 325A, 325B, 325C at a plurality of levels (e.g., depths 322, 324, 326) that are numbered from a deepest level (e.g., a first depth 322) to a highest level (e.g., a third depth 326). Each of the plurality of bins 325A, 325B, 325C may comprise summary data that corresponds to a respective portion of the sequencing data (e.g., the genome). The summary data may correspond to a given portion of sequencing data being requested by the user for display in a genome viewer (e.g., genome browser or other application). The summary data in each of the plurality of bins 325A, 325B, 325C may be calculated using the reads in the respective portion of the sequencing data (e.g., genome) that overlap the respective bin of the plurality of bins 325A, 325B, 325C. For VCF/gVCF file, the summary data in each of the plurality of bins may be calculated using the variants in the respective portion of the sequencing data. For BED files, the summary data in each of the plurality of bins may be calculated using a numerical value.

The number of bins 325A, 325B, 325C at each depth 322, 324, 326 may be calculated such that after the aggregate file 300 is generated, when the computing device attempts to find data corresponding to a specific genome location, the computing device may calculate a byte offset of the bins 325A, 325B, 325C corresponding to the desired depth and genome location. Calculating the byte offset associated with the desired depth and genome location may be faster than having to read an index file and look up the correct byte offset.

Each of the plurality of bins 325A, 325B, 325C may consume (e.g., occupy) an equal size in memory (e.g., such as the memory 204 shown in FIG. 2). For example, each of the plurality of bins 325A, 325B, 325C may comprise the same type(s) of summary data. The summary data may comprise a plurality of metrics 335 that summarize the reads in the respective bin of the plurality of bins 325A, 325B, 325C. The plurality of metrics 335 may comprise a mean mapping quality (e.g., mean MAPQ), a mean depth, an A proportion, a T proportion, a C proportion, and/or a G proportion.

The mean MAPQ may represent a mean of the sums of MAPQ of the proportion of the read that overlaps a respective bin of the plurality of bins 325A, 325B, 325C. The mean MAPQ may be determined from the BAM or SAM file. The BAM index file may be used to skip to an area of the BED file to identify the MAPQ scores from which to calculate the mean MAPQ.

The mean depth may be a mean mapped read depth that represents a sum of mapped read depths at a genomic position (e.g., a reference base position). The mean depth may be determined from the BAM or SAM file. For each read that is overlapping a region, the length of the read may be multiplied by the percentage that it overlaps the region and the result may be added to the total depth for that bin. For example, if a read is 150 base pairs long and 90% of it overlaps a bin, then the 135 base pair value may be added to the total depth of the bin. The total depth may be divided by the number of bases in the bin to get the mean depth.

The read depth may indicate how many reads detected a specific nucleotide. The read depth may represent a number of times a particular base is represented within each of the reads in the sequencing data. Sometimes the wrong base may be incorporated into a DNA fragment identified in the sequencing data. For example, a camera in the sequencing device may pick up the wrong signal, a read may be misplaced, or a sample may be contaminated to cause an incorrect base to be called in the sequencing data. By sequencing each fragment numerous times to produce multiple reads, there is a confidence or likelihood that identified variants are true variants and not artefacts from the sequencing process. The read depth represents the number of times each individual base has been sequenced or the number of reads in which the individual base appears in the sequencing data. The higher the read depth, the greater the level of confidence in variant calling. The read depth may be expressed as an average or percentage exceeding a cutoff over a set of intervals (such as exons, bases, genes, or panels). The read depth may be an indicator of the reliability of a base call. Low read depth may indicate that a specific region is poorly represented in the sample.

The A proportion may represent a proportion of A nucleotides (e.g., in a genomic region that corresponds to the respective one of the bins 325A, 325B, 325C). The T proportion may represent a proportion of T nucleotides (e.g., in a genomic region that corresponds to the respective one of the bins 325A, 325B, 325C). The C proportion may represent a proportion of C nucleotides (e.g., in a genomic region that corresponds to the respective one of the bins 325A, 325B, 325C). The G proportion may represent a proportion of G nucleotides (e.g., in a genomic region that corresponds to the respective one of the bins 325A, 325B, 325C). The A proportion, T proportion, C proportion, and/or G proportion may be determined from the BAM or FASTA file by counting the number of bases. The proportion of each nucleotide may be represented as a percentage or decimal value indicating the proportion of the nucleotide observed in the sequencing data. Each of the proportions may be calculated as a normalized count or a raw count. The count may be determined for the lowest level bin from the BAM or FASTA file and dividing the proportion by the number of reads. The count for each higher level bin may be determined by summing the counts of each child bin. After all the counts have been done for each bin, the proportions may be calculated by dividing the number of each nucleotide by the total number of nucleotides in the bin.

It should be appreciated that the plurality of metrics 335 is not limited to this list, rather the plurality of metrics 335 may comprise one or more other and/or alternate metrics that summarize the reads that overlap the respective bin (e.g., that are in a genomic region that corresponds to the respective one of the bins 325A, 325B, 325C).

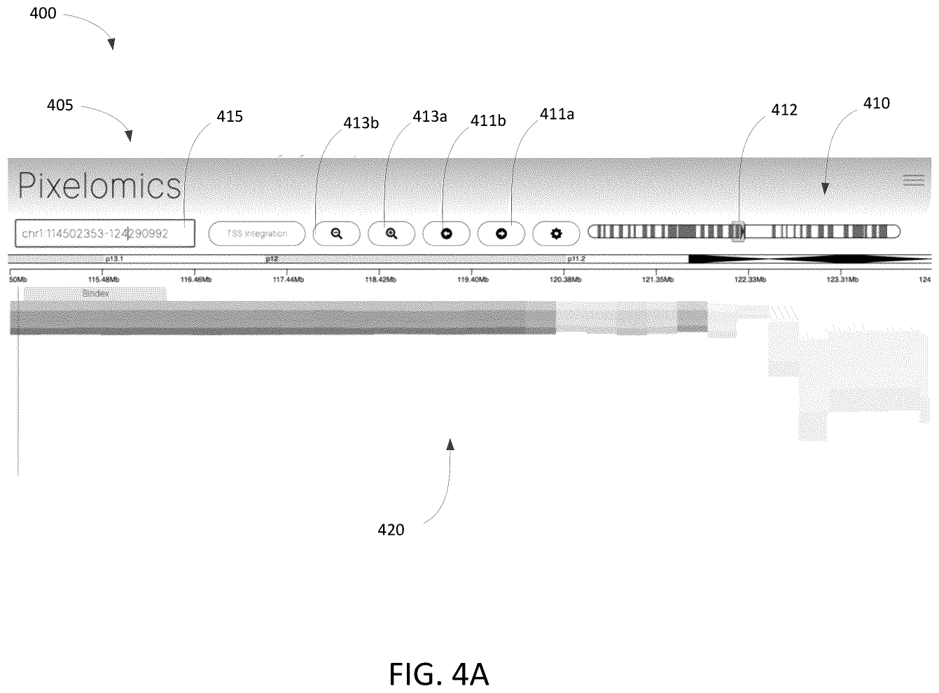

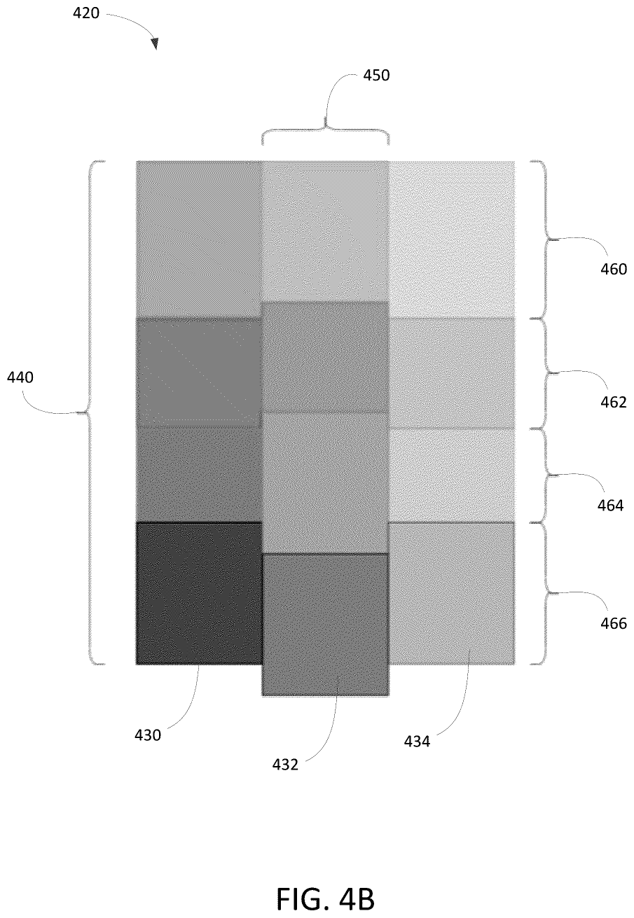

The bin list 304 may comprise a bin format 320. For example, the computing device may generate the aggregate file 300 based on the bin format 320. The computing device may determine the bin format 320 based on the genome data and/or one or more capabilities of a genome viewer (e.g., such as the aggregate viewer 400 shown in FIGS. 4A and 4B). For example, the computing device may determine how many depths (e.g., levels) of bins 325A, 325B, 325C and/or how may bins 325A, 325B, 325C at each depth to include in the aggregate file 300 (e.g., the bin format 320) based on the reference length of the genome. The plurality of bins 325A, 325B, 325C may be organized into the bin format 320. The bin format 320 may comprise one or more bins 325A, 325B, 325C for each of the plurality of depths 322, 324, 326. In the example aggregate file 300 shown in FIG. 3A, the first depth 322 may comprise a plurality of first bins 325A, a second depth 324 may comprise a plurality of second bins 325B, and the third depth 326 may comprise a third bin 325C. The first depth 322 may represent a lowest depth of the bin format 320, the second depth 324 may represent a middle depth of the bin format 320, and the third depth 326 may represent a highest depth of the bin format 320. For example, the highest depth (e.g., the third depth 326) may comprise summary data for the entire genome in a single bin (e.g., the third bin 325C). Each depth may include different levels of summary information for different portions of the sequencing data (e.g., the genome) that may be displayed together.

Each of the plurality of second bins 325B may summarize data (e.g., summary data) from a respective subset of the plurality of first bins 325A. For example, bin 9 may summarize the data in bin 0, bin 1, and bin 2, bin 10 may summarize the data in bin 3, bin 4, and bin 5, and bin 11 may summarize the data in bin 6, bin 7, and bin 8. The third bin 325C may comprise summary data for the plurality of second bins 325B. For example, bin 12 may summarize the data in bin 9, bin 10, and bin 11. The summary data for the plurality of first bins 325A may be calculated first. The summary data for the plurality of second bins 325B may be calculated using the summary data for the plurality of first bins 325A. For example, the summary data for bin 0, bin 1, and bin 2 may be summarized to generate the summary data for bin 9. The summary data for the third bin 325C may be calculated using the summary data for the plurality of second bins 325B. For example, the summary data for bin 9, bin 10, and bin 11 may be summarized to generate the summary data for bin 12.

The header 302 may comprise a plurality of header contents 310. The header contents 310 may include information that enables reading data from the aggregate file and/or the bins 325A, 325B, 325C. For example, the header contents 310 may include a name length, a genome name, a reference length, and/or a scale factor. The name length and/or the genome name may identify a genome associated with the sequencing data and the aggregate file 300. The scale factor may define how many bins 325A, 325B, 325C from a lower level are in each bin of a higher level. For example, the scale factor may define how many first bins 325A of the first depth 322 are summarized in each of the second bins 325B of the second depth 324 and/or how many second bins 325B of the second depth 324 are summarized by the third bin 326.

Table 1 includes example data for each of the bins 325A, 325B, 325C shown in FIG. 3A.

TABLE 1
Example Bin Data

| | Bin | Mean | Mean | | | | |
|---|---|---|---|---|---|---|---|
| Bins | No. | MAPQ | Depth | A Prop | T Prop | C Prop | G Prop |
| 325A | 0 | 59.23169 | 71.44488 | 0.31419 | 0.33244 | 0.16822 | 0.18513 |
| 325A | 1 | 57.59852 | 43.56742 | 0.27206 | 0.28902 | 0.20527 | 0.23362 |
| 325A | 2 | 58.79865 | 85.56521 | 0.29453 | 0.25273 | 0.22970 | 0.22303 |
| 325A | 3 | 57.98421 | 99.23475 | 0.24912 | 0.23322 | 0.27491 | 0.24274 |
| 325A | 4 | 59.04524 | 23.53262 | 0.27999 | 0.27123 | 0.22290 | 0.22588 |
| 325A | 5 | 59.49852 | 80.15426 | 0.25571 | 0.25785 | 0.25317 | 0.23326 |
| 325A | 6 | 58.93170 | 39.23169 | 0.25056 | 0.24997 | 0.24820 | 0.25125 |
| 325A | 7 | 57.84617 | 113.69802 | 0.27956 | 0.24690 | 0.22702 | 0.24650 |
| 325A | 8 | 55.48752 | 82.76492 | 0.25069 | 0.28134 | 0.22726 | 0.24069 |
| 325B | 9 | 58.54295 | 66.85917 | 0.29359 | 0.29140 | 0.20106 | 0.21393 |
| 325B | 10 | 58.84266 | 67.64054 | 0.26161 | 0.25410 | 0.25033 | 0.23396 |
| 325B | 11 | 57.42180 | 78.56488 | 0.26027 | 0.25940 | 0.23416 | 0.24615 |
| 325C | 12 | 58.26914 | 71.02153 | 0.27182 | 0.26830 | 0.22852 | 0.23134 |

Similar bin data may be generated for each layer of bins. The header contents 310 may include information that enables reading data from the aggregate file and/or the bins 325A, 325B, 325C in response to a request from a user input at a browser viewer.

FIG. 3B depicts another example bin format 350 for the aggregate file 300. The aggregate file 300 (e.g., the bin format 350) may be generated by a computing device (e.g., such as the client device 108, the server device 102, and/or the computing device 200 shown in FIGS. 1A and 2, respectively). The bin format 350 may comprise a plurality of bins 355A, 355B, 355C, 355D at a plurality of levels (e.g., depths 352, 354, 356, 358) that are numbered from a deepest level (e.g., a first depth 352) to a highest level (e.g., a fourth depth 358). Each of the plurality of bins 355A, 355B, 355C, 355D may comprise summary data that corresponds to a respective portion of the sequencing data (e.g., the genome). The summary data may be calculated for being displayed for a given portion of sequencing data being requested by the user for display in a genome viewer (e.g., genome browser or other application). The summary data in each of the plurality of bins 355A, 355B, 355C, 355D may be calculated using the reads in the respective portion of the sequencing data (e.g., genome) that overlap the respective bin of the plurality of bins 355A, 355B, 355C, 355D.

Each of the plurality of bins 355A, 355B, 355C, 355D may consume (e.g., occupy) an equal size in memory (e.g., such as the memory 204 shown in FIG. 2). For example, each of the plurality of bins 355A, 355B, 355C, 355D may comprise the same type(s) of summary data. The summary data may comprise the plurality of metrics 335 shown in FIG. 3A that summarize the reads in the respective bin of the plurality of bins 355A, 355B, 355C, 355D shown in FIG. 3B.

For example, the computing device may generate the aggregate file 300 based on the bin format 350. The computing device may determine the bin format 350 based on the genome data and/or one or more capabilities of a genome viewer (e.g., such as the aggregate viewer 400 shown in FIGS. 4A and 4B). For example, the computing device may determine how many depths (e.g., levels) of bins 355A, 355B, 355C, 355D to include in the aggregate file 300 (e.g., the bin format 320) and/or the number of bins 355A, 355B, 355C, 355D at each depth based on the reference length of the genome and/or how many reads are included in the genome data. For example, the computing device may determine a location in the aggregate file 300 of respective bins of the plurality of bins 355A, 355B, 355C, 355D that overlap a specific genomic region at a specific depth of the plurality of depth 352, 354, 356, 358 based on the size (e.g., in bytes) of each of the bins 355A, 355B, 355C, 355D, the scale factor, and a length of the genome.

The plurality of bins 355A, 355B, 355C, 355D may be organized into the bin format 350. The bin format 350 may comprise one or more bins 355A, 355B, 355C, 355D for each of the plurality of depths 352, 354, 356, 358. For example, the first depth 352 may comprise a plurality of first bins 355A, a second depth 354 may comprise a plurality of second bins 355B, a third depth 356 may comprise a plurality of third bins 355C, and a fourth depth 358 may comprise a fourth bin 355D. The first depth 352 may represent a lowest depth of the bin format 350, the second and third depths 354, 356 may represent middle depths of the bin format 350, and the fourth depth 358 may represent a highest depth of the bin format 350. For example, the highest depth (e.g., the third depth 358) may comprise summary data for the entire genome.

Each of the plurality of second bins 355B may summarize data (e.g., summary data) from a respective subset of the plurality of first bins 355A. Each of the plurality of third bins 355C may summarize data for a respective subset of the plurality of second bins 355B. The fourth bin 355D may summarize the data for the plurality of third bins 355C. The summary data for the plurality of first bins 355A may be calculated first. The summary data for the plurality of second bins 355B may be calculated using the summary data for the plurality of first bins 355A. The summary data for the plurality of third bins 355C may be calculated using the summary data for the plurality of second bins 355B. The summary data for the fourth bin 355D may be calculated using the summary data for the plurality of third bins 355C. The summary data in each of the bins at each level may be separately stored in memory at the computing device for being accessed in response to a user request to display information related to a different portion of the sequencing data (e.g., the genome) (e.g., zoom in or zoom out of different portions of the sequencing data).

A target depth 360 (e.g., the third depth 356 in the example shown in FIG. 3B) may be determined for a selected genomic region. The selected genomic region may be defined by a pair of genomic coordinates. The displayed portion of summary data may be associated with one or more of the bins at the target depth 360 that corresponds with the selected genomic region. Reading from the aggregate file 300 may comprise determining the target depth 360 to read from. The computing device may locate the target bin(s) 365 associated with the selected genomic region after determining the target depth 360. The computing device may calculate the bin size at the target depth 360. The computing device may then determine which bins (e.g., target bins 365) at the target depth 360 overlap the selected genomic region, for example, based on the bin size at the target depth 360.

The target bin(s) 365 at the target depth 360 may be determined based on the selected genomic region and the calculated bin size. For example, the selected genomic region may be converted to genomic position(s). For example, the target bin(s) 365 at the beginning and end of the selected genomic region may be calculated using the genomic positions that correspond with the beginning of the selected genomic region and the end of the selected genomic region, respectively.

Table 2 includes example data for each of the bins 355B, 355C, 355D shown in FIG. 3B. The data for the plurality of first bins 355A is not shown in Table 2 for simplicity purposes. However it should be appreciated that each of the plurality of second bins 355B may be associated with (e.g., comprise averages of) three of the plurality of second bins 355 each having a bin size of 17,450. The bin size for the plurality of second bins 355B may be 52,350. The bin size for the plurality of third bins 355C may be 157,050. The bin size for the fourth bin 355D may be 420,413. The beginning and end of each bin is calculated from the start of the genome (position 1), the bin size, and how many bins preceded the bin in question.

TABLE 2
Example Bin Data

| | | | Mean | Mean | | | | |
|---|---|---|---|---|---|---|---|---|
| Bin | Begin | End | MAPQ | Depth | A Prop | T Prop | C Prop | G Prop |
| 355B | 0 | 173 | 59.23169 | 71.44488 | 0.31419 | 0.33244 | 0.16822 | 0.18513 |
| 355B | 173 | 52703 | 57.59852 | 43.56742 | 0.27206 | 0.28902 | 0.20527 | 0.23362 |
| 355B | 52703 | 105233 | 58.79865 | 85.56521 | 0.29453 | 0.25273 | 0.22970 | 0.22303 |
| 355B | 105233 | 157763 | 57.98421 | 99.23475 | 0.24912 | 0.23322 | 0.27491 | 0.24274 |
| 355B | 157763 | 210293 | 59.04524 | 23.53262 | 0.27999 | 0.27123 | 0.22290 | 0.22588 |
| 355B | 210293 | 262823 | 59.49852 | 80.15426 | 0.25571 | 0.25785 | 0.25317 | 0.23326 |
| 355B | 262823 | 315353 | 58.93170 | 39.23169 | 0.25056 | 0.24997 | 0.24820 | 0.25125 |
| 355B | 315353 | 367883 | 57.84617 | 113.69802 | 0.27956 | 0.24690 | 0.22702 | 0.24650 |
| 355B | 367883 | 420413 | 55.48752 | 82.76492 | 0.25069 | 0.28134 | 0.22726 | 0.24069 |
| 355C | 0 | 105233 | 58.54295 | 66.85917 | 0.29359 | 0.29140 | 0.20106 | 0.21393 |
| 355C | 105233 | 262823 | 58.84266 | 67.64054 | 0.26161 | 0.25410 | 0.25033 | 0.23396 |
| 355C | 262823 | 420413 | 57.42180 | 78.56488 | 0.26027 | 0.25940 | 0.23416 | 0.24615 |
| 355D | 0 | 420413 | 58.26914 | 71.02153 | 0.27182 | 0.26830 | 0.22852 | 0.23134 |

Similar bin data may be generated for each layer of bins. The header contents 310 may include information that enables reading data from the aggregate file and/or the bins 325A, 325B, 325C in response to a request from a user input at a browser viewer.

In an example, the selected genomic region may be represented as chr3:235595-335695. The beginning of the selected genomic region may be chr3:235595 and the end of the selected genomic region may be chr3:335695. The computing device may determine whether the selected genomic region overlaps one or two bins at the first depth 352, the second depth 354, or the third depth 356. In this example, the selected genomic region, chr3:235595-335695, may overlap at least a portion of two of the plurality of third bins 355C (e.g., each of the target bins 365) at the third depth. For example, the beginning of the selected genomic region, chr3:235595-335695, may correspond with (e.g., be located within) a first one of the target bins 365 and the end of the selected genomic region, chr3:235595-335695, may correspond with (e.g., be located within) a second one of the target bins 365.

It should be appreciated that although the example bin formats 320, 350 shown in FIGS. 3A and 3B depict three and four depths, respectively, the bin formats 320, 350 of the aggregate file 300 may comprise more than four or less than three depths. It should also be appreciated that although the example bin format 320 shown in FIG. 3A depicts 9 bins at the lowest depth (e.g., the first depth 322) and the example bin format 350 shown in FIG. 3B depicts 27 bins at the lowest depth (e.g., the first depth 352), the aggregate file 300 (e.g., a bin format of the aggregate file 300) may comprise more than 27 bins, less than 9 bins, or between 9 and 27 bins at the lowest depth. The aggregate file 300 may be generated and stored at a computing device (e.g., a client device or one or more server devices) for accessing the data in one or more bins in one or more levels in response to requests from a user to display information in a genome viewer (e.g., genome browser or other application).

A genome viewer (e.g., genome browser or other application) may be configured to display data associated with a selected region of genomic data. The genome viewer may enable user selection of a portion of a genome (e.g., a genomic region) at a zoom level. The genome viewer may send a request for the selected portion of the genome (e.g., a genomic region). For example, the genome viewer may be operating on a client device and may send a request to local memory or to one or more remote computing devices (e.g., one or more server devices). The genome viewer may receive and display summary data stored in an aggregate file that corresponds with the selected genomic region at the zoom level. The genome viewer may display the summary data from the aggregate file using one or more display conditions to indicate relative differences between portions of the summary data. For example, a computing device (e.g., such as the client device 108, the server device 102, and/or the computing device 200 shown in FIGS. 1A and 2, respectively) may separate the summary data into adjacent portions that are then stored in the aggregate file for various zoom levels. The genome viewer may be configured to display summary information at any zoom level up to the entire genome, which provides a consistent display of data as the range (e.g., genomic region and/or zoom level) changes.

FIG. 4A depicts an example genome viewer that is operating as an aggregate viewer 400. FIG. 4B depicts a partial detailed view of a selection display area 420 of the aggregate viewer 400. The aggregate viewer 400 shown in FIG. 4A may include a genome viewer or genome browser. The aggregate viewer 400 may comprise a user interface 405 that is configured to enable display and visualization of summary data associated with a genome that is stored in an aggregate file (e.g., such as the aggregate file 300 shown in FIG. 3A). The aggregate viewer 400 may comprise a chromosome ideogram 410. The chromosome ideogram 410 may represent a condensed view of a chromosome within the genome. The aggregate viewer 400 may comprise a genomic region selection indicator 412. The genomic region selection indicator 412 may indicate which portion of the genome has been selected (e.g., by a user). For example, the genomic region selection indicator 412 may be moved by a user to select a desired portion of the genome (e.g., a genomic region). The portion of the genome that has been selected may be defined by a pair of genomic coordinates. The portion of the genome that has been selected may correspond with a plurality of chromosomes. The user may select a position to the left or right on the chromosome ideogram 410 to scroll to different portions of the genome.

The aggregate viewer 400 (e.g., the user interface 405) may comprise a text box 415. The text box 415 may enable input of a genomic region (e.g., chromosome range). The text box 415 may display a selected genomic region (e.g., chromosome range) that corresponds with the genomic region selection indicator 412. For example, the text box 415 may display the pair of genomic coordinates that defines the genomic region. In response to entry of a genomic region in the text box 415 and actuation of a button or other input from the user, the aggregate viewer 400 may send a request for the summary data for the defined genomic region. The genomic region selection indicator 412 may be updated to indicate the genomic region in the text box 415. The user may zoom in or out of different portions of the genome by selecting the zoom in button 413a or the zoom out button 413b, respectively. The aggregate viewer 400 may zoom in or out by a predefined amount in response to selection of the zoom buttons 413a, 413b. The user may scroll to earlier or later genomic regions by selecting the scroll button 411b or the scroll button 411a, respectively. The aggregate viewer 400 may scroll by a predefined amount in response to selection of the scroll buttons 411a, 411b. In response to the selection of the zoom buttons 413a, 413b and/or the scroll buttons 411a, 411b, the aggregate viewer 400 may send a request for the summary data for the defined genomic region. The text box 415 and/or the genomic region selection indicator 412 may be updated to indicate the defined genomic region in response to the selection of the zoom buttons 413a, 413b and/or the selection of the scroll buttons 411a, 411b.

The aggregate viewer 400 (e.g., the user interface 405) may comprise a selection display area 420. The selection display area 420 may display summary data associated with the sequencing data for the selected portion of the genome. For example, the selection display area 420 may display summary data for bins 430, 432, 434 (e.g., at a target depth) shown in FIG. 4B that overlap the selected portion of the genome. Each of the bins 430, 432, 434 displayed in the selection display area 420 may define a bin length 450 that is the same across the target depth. For example, each of the bins 430, 432, 434 at the target depth may have the same bin length 450. The bin length 450 may be determined based on the size of the genomic data and the depth of the bins 430, 432, 434.

The summary data may be displayed using one or more display conditions. The one or more display conditions may represent relative differences in the summary data between reads within the one or more of the bins 430, 432 434. The one or more display conditions comprise color, opacity, and/or height, for example, as shown in FIG. 4B. Each display condition may correspond to a different type of summary data. An opacity of the bins 430, 432, 434 may represent a mean quality of the reads associated with the portion of the genomic region within that respective one of the bins 430, 432, 434. For example, the opacity of the bin representation in the displayed portion of summary data may represent an average read quality for the reads associated with the portion of the genomic region within that bin. A total height 440 of the bin representation in the displayed portion of summary data may indicate the average depth of the reads associated with portion of the genomic region within that bin of the bins 430, 432, 434.

Color may be used to represent the nucleotide proportions 460, 462, 464, 466 in each of the bins 430, 432, 434. For example, each nucleotide base may be assigned a color for the entire data set and the relative height of each color in a bin may represent the proportions 460, 462, 464, 466 of the respective nucleotide bases in that bin. A first proportion 460 may represent the proportion of A bases in each respective one of the bins 430, 432, 434. A second proportion 462 may represent the proportion of T bases in each respective one of the bins 430, 432, 434. A third proportion 464 may represent the proportion of C bases in each respective one of the bins 430, 432, 434. A fourth proportion 466 may represent the proportion of G bases in each respective one of the bins 430, 432, 434. It should be appreciated that the display conditions are not limited to these examples, rather the display conditions may include one or more other physical characteristics such as shading, hashing, integers, descriptions, patterns, shapes, and/or the like.

Table 3 depicts example aggregate viewer data used by the aggregate viewer 400 to display the partial detailed view of the selection display area 420 shown in FIG. 4B. Some of the details illustrated on the display area 420, such as opacity and height, may be calculated from the aggregate viewer data stored in a file, rather than being stored in the file itself.

TABLE 3
Example Aggregate Viewer Data

| Bin | Mean | Mean | | | | | | |
|---|---|---|---|---|---|---|---|---|
| No. | MAPQ | Depth | A Prop | T Prop | C Prop | G Prop | Opacity | Height |
| 430 | 59.9999 | 41.3730 | 0.26670 | 0.01790 | 0.29186 | 0.26243 | 0.699 | 1041.37 |
| 432 | 60 | 44.3027 | 0.26162 | 0.22399 | 0.27918 | 0.23521 | 0.700 | 1044.30 |
| 434 | 59.41176 | 40.8000 | 0.25174 | 0.24605 | 0.24257 | 0.25964 | 0.694 | 1040.80 |

The aggregate viewer data may be used by the aggregate viewer 400 to generate a display. Since the beginning and end of each bin are known, the aggregate viewer 400 may determine the x coordinate and the width of rectangles that may be drawn for each bin on the display. The mapQ value may be divided by 60 to get the opacity. The mean depth may be used to calculate the total height of the rectangle to draw based on the mean depth for the whole genome and the height of the canvas in pixels. The height of each rectangle for A, C, T, G may be a fraction of the calculated total height. For example, if total height for bin 430 is 100 pixels (based on 41.373 compared with the mean depth across the whole genome, and the height of the canvas), then the height of A in bin 430 may be 26.6 pixels

An intermediate file (e.g., aggregate file) may be created from received genome data. The intermediate file may separate the received genome data into equal sized portions at various levels (e.g., zoom levels). The intermediate file may summarize the genome data associated with each of the portions to enable visualization of relevant data for those portions of the genome selected by the user in the genome viewer. The intermediate file may summarize genome data for display at respective zoom levels selectable in the genome viewer (e.g., aggregate viewer). The genome viewer may be configured to display the summary data associated with a selected region of genomic data. The genome viewer may receive a selection of a genomic region by a user. The genome viewer may identify summary data stored in an aggregate file to display based on the selected region of genomic data. The data that is provided in the genome viewer may be different at different predefined zoom levels. The genome viewer may be capable of displaying summary data at low zoom levels (e.g., even when the selected region of genomic data is substantially the entire genome). The genome viewer may be capable of displaying more specific summary data at higher zoom levels.

In one example, the summary data in the bins may provide a first level of detail that may be displayed in the genome viewer. If the user zooms to a certain level to focus in on a smaller portion of the chromosome, the individual non-summary files themselves (e.g., BED files, FASTA files, BAM files, etc.) may be accessed to provide additional levels of detail related to the coordinates being viewed in the genome viewer. The genome viewer may send a request for sequencing data for a genomic region and the binned summary data may be retrieved (e.g., by the one or more server devices) without using an index file, directly from the aggregated summary file, or using the original index file (e.g., .bai index file) from the original data file (e.g., .bam file). In one example, more specific data may be accessed from the individual non-summary files themselves (e.g., BED files, FASTA files, BAM files, etc.) when the zoom level reaches a threshold. The individual non-summary files (e.g., BED files, FASTA files, BAM files, etc.) may be accessed when a first zoom threshold is reached and/or some of the data may be filtered out to limit the amount of data being retrieved. The data that is filtered out may be based on additional thresholds for each type of data in the non-summary file. For example, the individual non-summary files may be accessed from the BED file and reads may be filtered out that have a mapQ of less than 60. Additionally, or alternatively, a minimum amount of data or data types may be retrieved per entry from the non-summary files. For example, for a given read, the genome viewer may return a subset of data types (e.g., chromosome, position, and/or CIGAR string) from the total data types stored in the non-summary files. From the subset of data types, the genome viewer may display a subset of information, such as the reads the base mismatches, insertions, and/or deletions. The zoom level may be increased to additional zoom level thresholds, such as a second zoom level threshold. In one example, when the second zoom level threshold (e.g., a region of 1000 bases) is reached, each of the reads in a region may be retrieved and/or the original data (e.g., BED files, FASTA files, BAM files, etc.) from the non-summary files may be displayed for each read. In an example, between 1000 bases and 100,000 bases, the first threshold may be met such that a filtered, minimal data may be displayed. A zoom level above 100,000 bases may be set such that the summary data (e.g., aggregated binned data) may be displayed.

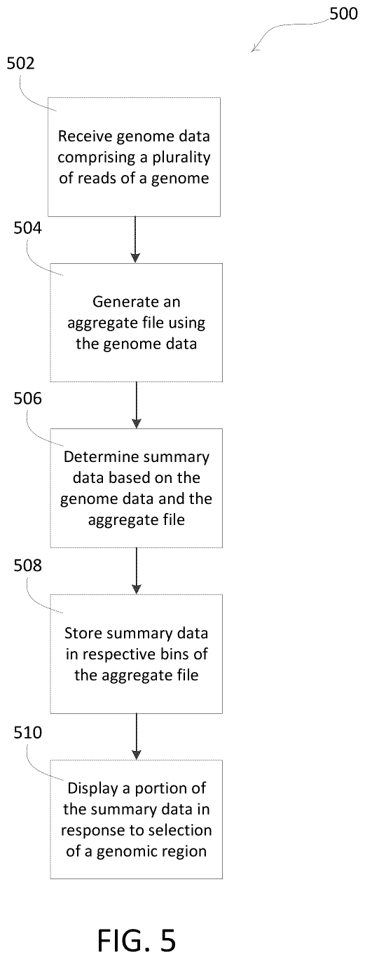

FIG. 5 is a flowchart depicting an example process 500 for generating an aggregate file and displaying a portion of summary data stored in the aggregate file. The process 500 may enable displaying relevant summary data associated with a selected portion of a genome. For example, the process 500 may be used to display summary data associated with the selected portion of a genome. One or more portions of the process 500 may be performed by a genome viewer (e.g., genome browser or other application). One or more portions of the process 500 may be performed by one or more computing devices (e.g., such as the client device 108, the server device 102, and/or the computing device 200 shown in FIGS. 1A and 2, respectively). One or more portions of the process 500 may be stored in memory as computer-readable or machine-readable instructions that may be executed by a processor of the one or more computing devices. Though portions of the process 500 may be described herein as being performed by a single computing device, the process 500, or portions thereof, may be distributed across multiple devices, such as a client computing device (e.g., such as the client device 108 shown in FIG. 1A), a genotyping device (e.g., such as the sequencing device 114 shown in FIG. 1A), and/or one or more server computing devices (e.g., such as the server device(s) 102 shown in FIG. 1A).

The process 500 may begin at 502. As shown in FIG. 5, at 502 the computing device may receive genome data associated with a genome. For example, the genome data may comprise genome sequencing data. The genome data may be received in an alignment map file. The alignment map file may be a binary alignment map (BAM) file or a sequence alignment map (SAM) file. The genome data may be received in a FASTA or FASTQ file. The genome data may be received in a BED file and/or a BedGraph file. The genome data may include variant calling data received in a VCF file and/or a gVCF file.

At 504, the computing device may generate an aggregate file (e.g., such as the aggregate file 300 shown in FIGS. 3A and 3B) using the received genome data. The aggregate file may comprise a plurality of bins at a plurality of depths. Each of the plurality of bins may be associated with a subset of the reads, variants, and/or annotated regions in the genome data. The computing device may analyze the BAM file, the SAM file, the BED file, and/or the VCF/gVCF when generating the aggregate file. For example, the computing device may determine how many depths to include in the aggregate file and/or how many bins to include in each depth of the aggregate file based on a reference length of the genome. The reference length of the genome may be stored within the BAM file or the SAM file. Each of the plurality of bins may overlap a respective portion of the genome that includes the respective subset of reads. A read that overlaps two of the plurality of bins may be assigned to one of the two bins based on how much it overlaps each of the two bins. The plurality of bins may comprise a first set of bins at a first depth, a second set of bins at a second depth, and a third set of bins at a third depth. Each of the second set of bins may comprise a plurality of the first set of bins at the first depth. Each of the third set of bins may comprise a plurality of the second set of bins at the second depth. For example, each of the plurality of bins at a respective depth (e.g., at each depth except the lowest depth) may cover a subset of the bins of the next highest depth. Stated differently, a subset of bins at a lower depth may merge into a bin at the next highest depth.

The aggregate file may comprise a header that indicates one or more of a name length, a genome name, a reference length, or a scale factor. The scale factor may indicate how many bins of a respective set of bins at a proximate depth are comprised within a respective one of the plurality of bins. The proximate depth may be defined as the next lowest depth. The bins in the aggregate file may be generated based on the reference length of the genome, the scale factor, and a minimum bin size. The proximate depth may be generated first, as a single bin size may be determined from the reference length for the genome. For example, the scale factor may indicate how many bins of a lower depth (e.g. a next lower depth) combine into (e.g., merge into) a respective one of the plurality of bins at a next depth (e.g., next higher depth). The scale factor may indicate how many bins of the second set of bins are comprised within the third set of bins and how many bins of the first set of bins are comprised within the second set of bins. The name length and the genome name may identify the genome. For example, the name length and the genome name may comprise genome identifiers. The computing device may determine how many layers (e.g., depths) the aggregate file should have based on the reference length and/or the scale factor. For example, the computing device may determine a minimum depth and a maximum depth for the aggregate file based on the reference length and/or the scale factor.

The bins may be generated individually for each chromosome for the whole genome using the reference length of the chromosome, the scale factor, and the minimum bin size. The bins of summary data for the aggregate file may be generated for each chromosome because chromosomes are not contiguous (e.g., as they may be represented contiguously in silico). So each chromosome may be binned at the proximate depth or the next lowest depth and the scaling factor may be used to generate higher level bins by dividing the summary data by the number of bins, as further described herein.

At 506, the computing device may determine summary data for respective reads associated with one or more respective portions of the genome covered by respective bins of the plurality of bins based on the received genome data and the aggregate file. The summary data may comprise one or more of an average quality, an average depth, or one or more nucleotide proportions. For example the average quality may represent a mean mapping quality for the reads associated with the respective portion of the genome. The average depth may represent a mean of mapped read depths for the reads associated with the respective portion of the genome. The one or more nucleotide proportions may represent how many A bases, T bases, C bases, and G bases are within the reads associated with the respective portion of the genome. The computing device may read (e.g., analyze) the BAM file to identify the respective reads, for example, when determining the summary data. For example, the computing device may analyze the reads associated with the respective portions of the genome to calculate the summary data for each of the plurality of bins.

The computing device may determine summary data for each of the depths in successive order. For example, the computing device may first determine summary data for each of the bins at the lowest depth of the aggregate file. The computing device may then determine summary data for successive depths of the aggregate file using the determined summary data for an adjacent depth (e.g., previous depth). For example, the computing device may determine a first set of summary data for the first set of bins at the first depth. The computing device may determine a second set of summary data for the second set of bins at the second depth using the determined first set of summary data for the first set of bins. The computing device may determine a third set of summary data for the third set of bins at the third depth using the determined second set of summary data for the second set of bins.

At 508, the computing device may store the summary data for the respective reads in the respective bins of the plurality of bins that cover the respective portions of the genome associated with the respective reads. The second set of bins (e.g., each of the second set of bins) comprises summary data associated with a plurality of the first set of bins at the first depth. Each of the third set of bins comprises summary data associated with a plurality of the second set of bins at the second depth. Each of the bins at a specific depth may comprise summary data of an equal portion of the genome. For example, each of the first set of bins at the first depth comprise summary data for an equal portion of the genome having a first size, each of the second set of bins at the second depth may comprise summary data for an equal portion of the genome having a second size, and each of the third set of bins at the third depth may comprise summary data for an equal portion of the genome having a third size. Each of the plurality of bins may occupy an equal sized space of memory. The space of memory that is occupied by each of the plurality of bins may depend on a number of discrete variables comprised within the summary data.

At 510, the computing device may display a portion of the summary data in response to a selection of a genomic region by a user. The selected genomic region may be defined by a pair of genomic coordinates. For example, the computing device may determine that the user selected the genomic region. The computing device may identify the summary data associated with the selected genomic region. The displayed portion of summary data may be associated with one or more of the bins of the plurality of bins that correspond with the genomic region selected by the user. Reading from the aggregate file may comprise determining a target depth to read from. The computing device may then determine which bins at the target depth overlap selected genomic region. The computing device may locate the target bins associated with the selected genomic region after determining the target depth. The computing device may calculate the bin size at the target depth, for example, using Equation 1.

$$Bin Size = \frac{Reference Length}{Scale {Facfor}^{Target Depth}} \qquad ( Eq . 1 )$$

The target bin(s) may then be calculated based on the selected genomic region and the calculated bin size. For example, the selected genomic region may be converted to genomic position(s). The target bin(s) may be determined using Equation 2. For example, the target bin at the beginning and end of the selected genomic region may be calculated using the genomic positions that correspond with the beginning of the selected genomic region and the end of the selected genomic region, respectively.

$$Target Bin = \frac{Genomic Position}{Bin Size} \qquad ( Eq . 2 )$$

The computing device may then calculate an offset to the target depth, for example, using Equation 3.

$$Offset To Target Depth = {\sum }_{d = Target Depth + 1}^{Max Depth} Scale {Factor}^{d} \qquad ( Eq . 3 )$$

The computing device may determine which bytes to seek, for example, using Equation 4.

$$Bytes to Seek = ( ( Offset To Target Depth + Start Bin ) * Bytes per Bin ) + Header Size \qquad ( Eq . 4 )$$

For example, Equations 1-4 may be used to query the aggregate file (e.g., without using an index) to display a portion of summary data that corresponds with the genomic region selected. The portion of summary data may be displayed using one or more display conditions. The one or more display conditions may represent relative differences in the summary data between reads within the one or more bins of the displayed portion. The one or more display conditions comprise color, opacity, and/or height, for example, as shown in FIG. 4B. An opacity of the bin may represent a mean quality of the bin. For example, the opacity of the bin representation in the displayed portion of summary data may represent an average read quality for the reads associated with the portion of the genomic region within that bin. A total height of the bin representation in the displayed portion of summary data may indicate the average depth of the reads associated with portion of the genomic region within that bin. Color may be used to represent the nucleotide proportions in each bin. For example, each nucleotide base may be assigned a color for the entire data set and the relative height of each color in a bin may represent the proportion of the respective nucleotide bases in that bin. It should be appreciated that the display conditions are not limited to these examples, rather the display conditions may include one or more other physical characteristics such as shading, hashing, integers, descriptions, patterns, shapes, and/or the like.

The displayed portion of summary data may correspond with a depth of the plurality of depths. The computing device may determine the depth for the displayed portion of summary data based on the genomic region selected by the user. For example, the computing device may determine one or more bins at the determined depth that overlap the genomic region selected by the user. The computing device may convert a genomic region selected by the user to a location in the aggregate file. For example, the computing device may identify the location in the aggregate file that corresponds to the genomic region selected by the user. The location in the aggregate file may comprise a specific bin of the plurality of bins at a specific depth of the plurality of depths. For example, the computing device may identify the specific bin at the specific depth for the location based on a size of the location.

The process 500 (e.g., one or more portions of the process 500) may be repeated as a zoom level and/or selected genomic region changes. The user may zoom at any level up to the entire genome. For example, the displayed portion of summary data may be updated as the user zooms (e.g., at any level up to the entire genome) and/or changes the selected genomic region.

A genome viewer may be configured to display data associated with a selected region of genomic data. The genome viewer may receive a selection of a genomic region by a user. The computing device accessing the data for display by the genome viewer may identify whether to display summary data stored in an aggregate file or genomic data stored in the original file, for example, based on the selected region of genomic data. The genome viewer may be capable of displaying summary data from the aggregate file at lower zoom levels (e.g., when a zoom level is less than or equal to a predetermined threshold) and genomic data from the original file at higher zoom levels (e.g., when the zoom level is greater than the predetermined threshold). In an example, the genome viewer may be capable of displaying summary data when the selected region of genomic data is substantially the entire genome. When the zoom level is greater than the predefined threshold, the computing device providing the data to the genome viewer may access the BAM file (e.g., via a BAM index file) to provide additional information for the smaller section of the genome.

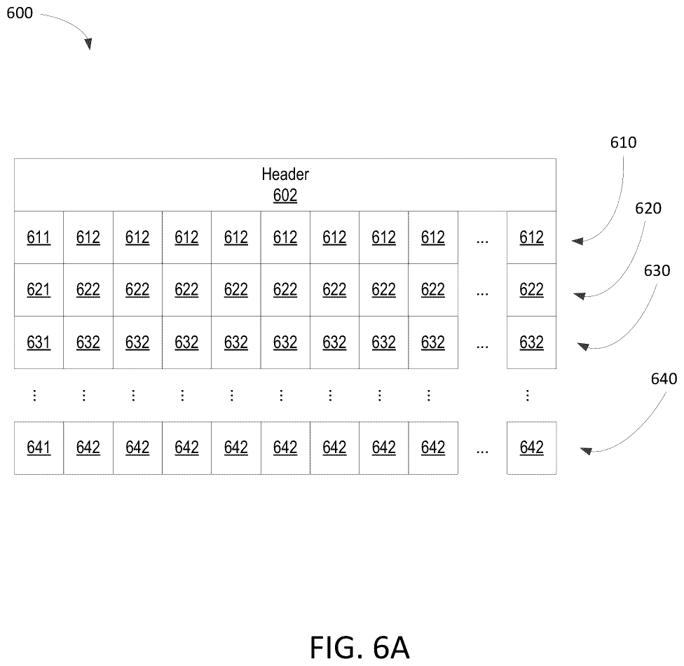

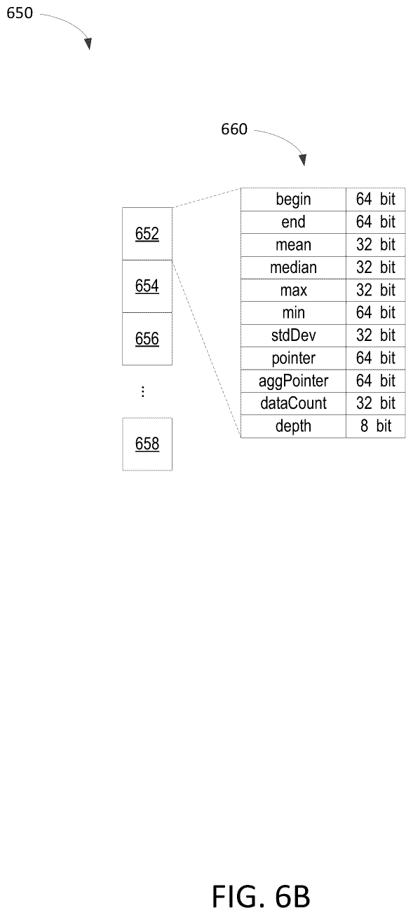

FIG. 6A is a diagram depicting an example format of an index file 600 that may be used for retrieving summary data. The index file 600 may be used when retrieving summary data from the aggregate file, or the summary data may be retrieved by a direct lookup of coordinates. The aggregate file may include a header containing the genome name, length, and/or scale factor, which may be used for calculating the byte offset to look in the file. The user of the index file 600 may avoid reading other data in the aggregate file that is outside of the location indicated by the index file 600. The index file 600 may comprise a header 602, a plurality of level blocks 611, 621, 631, 641, and a plurality of bins 612, 622, 632, 642. The header 602 may include a genomeString field, an aggregate code field, and/or a number of levels field. The genomeString field may indicate a name of the reference genome. The genome string may comprise 4 characters that represent the name of the reference genome. For example, for the reference genomes hg19, hg38, grch37, and grch38, the corresponding genome string for each would be hg19, hg38, gr37, and gr38, respectively. Each named reference genome may specify differing lengths for each chromosome. For example, chromosome 1 in hg19 reference genome may be 249250621 nucleotides (e.g., bases) long and chromosome 1 in hg38 reference genome may be 248956422 nucleotides (e.g., bases) long. Whenever reads are aligned they are aligned with respect to a given reference genome. The lengths of the chromosomes may be used to determine location on a display screen. The aggregate code field may indicate the type of summary performed. For example, the aggregate code may indicate a mean aggregation method, a median aggregation method, a maximum aggregation method, a minimum aggregation method, a standard deviation aggregation method, a box aggregation method, a general feature format (gff) aggregation method, or an RNA aggregation method. The number of levels field may indicate how many levels of bins are in the index file 600. Each of the plurality of level blocks 611, 621, 631, 641 may include a pointer and a bin number indicator. The pointer may comprise a virtual pointer that points to a string in memory of the aggregate file (e.g., such as the aggregate file 600). For example, the pointer may indicate a location in the aggregate file for a respective zoom level. The bin number indicator may indicate how many bins are at the respective level (e.g., associated with a respective zoom level) in the index file 600.

Each of the plurality of bins 612, 622, 632, 642 may comprise a begin indicator, an end indicator, a file indicator, and a pointer indicator. The begin indicator may comprise the genomic coordinates that represent a start location of the respective bin. The end indicator may comprise the genomic coordinates that represent an end location of the respective bin. The file indicator may indicate which file to look in for the data associated with the respective bin. For example, the file indicator may indicate whether to look in the aggregate file or the original file for the data. The file indicator may indicate whether to retrieve the data from the aggregate file or the original file. The pointer indicator may comprise a virtual pointer into the aggregate file or the original file.

In the example index file 600 shown in FIG. 6A, a first level 610 may comprise a plurality of first bins 612, a second level 620 may comprise a plurality of second bins 622, a third level 630 may comprise a plurality of third bins 632, a fourth level 640 may comprise a plurality of fourth bins 642. Although FIG. 6A depicts the index file 600 having more than 4 levels, it should be appreciated that the index file 600 may also have 4 or less levels. The first level 610 may comprise a first level block 611, the second level 620 may comprise a second level block 621, the third level 630 may comprise a third level block 631, and the fourth level 640 may comprise a fourth level block 641.

FIG. 6B is a diagram depicting an example format of an aggregate file 650. The aggregate file 650 may be configured for use with an index file (e.g., such as the index file 600 shown in FIG. 6A). The aggregate file 650 may be configured for use without an index file (e.g., and may not use a pointer, aggPointer, and/or other values used for reference by the index file).

The aggregate file 650 may be preconfigured from the FASTA file, FASTQ file, BAM file, the SAM file, VFC, gVCF, and/or the BED file (e.g., with or without the corresponding BED index file) for responsive access to requests for the summary data from the genome viewer. The aggregate file 650 may include statistics-based information, such as a mean, a max, a min, a median, or a standard deviation for the information in each bin. The aggregate file 650 may comprise a plurality of (e.g., a list of) bins 652, 654, 656, 658. For example, the aggregate file 650 may not include a header or any sections. Each of the plurality of bins 652, 654, 656, 658 may comprise a data block 660. The data block 660 may comprise a begin field, an end field, a mean field, a median field, a max field, a min field, a standard deviation (stdDev) field, a pointer field, an aggregate pointer (aggPointer) field, a data count field, and/or a depth field. The begin field may indicate genomic coordinates associated with a start of the respective bin. The end field may indicate genomic coordinates associated with an end of the respective bin. The mean field may indicate a mean value associated with the data within the respective bin. The median field may indicate a median value associated with the data within the respective bin. The max field may indicate a maximum value associated with the data within the respective bin. The stdDev field may indicate a standard deviation associated with the data within the respective bin. The pointer field may indicate a pointer associated with the data within the respective bin. The aggPointer field may indicate an aggregate pointer associated with the data within the respective bin. For example, the aggPointer field may be a pointer into the aggregate file that points to a beginning of a line (e.g., beginning of a bin) in the aggregate file. The pointer field may include a numerical byte offset to the first line in the non-summary compressed BED file that overlaps the bin. For example, the pointer field may include a byte offset to go to the non-summary file and identify the data that went into that bin, and seek to that pointer value. The data count field may represent how many data points from the original file were used to generate the data in the respective bin. In an example where the mean is calculated as 5 and the original file had values of [3,5,5,4,5,6,7] in the same genomic region of that respective bin, then those 7 values were used to generate the mean of 5. Thus, the data count for that respective bin would be 7. The depth field may indicate a depth associated with the respective bin. For example, a first bin 652 may be at a first depth (e.g., level), a second bin 654 may be at a second depth, a third bin 656 may be at a third depth, and a fourth bin 658 may be at a fourth depth.

For BED files, for each bin the begin field and/or the end field may be calculated, as described herein. For example, the length of the genome and the minimum bin size may be determined. The number of layers of bins and/or a size (e.g., in nucleotide bases) of each bin at each layer may be determined. Using the number of layers of bins and/or the bin size, when given a genomic coordinate range, the depth of the bins and/or the bins to retrieve may be calculated. As the layout of the aggregate file, the structure, and/or the size (e.g., in bytes of each bin) can be determined, the byte offset into the aggregate file may be calculated to get to the first bin including data to be displayed. The byte offset may be used to start reading bins until a bin is identified that doesn't overlap the region of the query for being displayed.

The mean field, median field, min field, max field, and/or stdDev field may be calculated from the specified column of interest in the non-summary BED file or BedGraph file. For example, if a BED file has 5 columns of data types (e.g., chr, begin, end, quality, and allele fraction), the user may specify to aggregate column 5 (e.g., allele fraction) then each of the rows that overlap the bin would be used to calculate the values of the mean field, the median field, the min field, the max field, and/or the stdDev field from column 5, assuming that each row has a valid numerical value. The depth field may be a value indicating the depth of the bin. The data count field may indicate how many lines (e.g., genomic regions) in the BED file overlapped the bin.

The aggregate file 650 may include count-based information, such as an aggregation of object counts for the information in each bin. For example, the aggregate file 650 may include an aggregate number of variants in a bin. The number of variants may be aggregated based on a number of single nucleotide polymorphisms (SNPs), structural variants (SVs) (e.g., insertions or deletions), and/or copy number variants (CNVs) identified for the bin. The SNPs, SVs, and/or CNVs may be determined or read from the VCF or gVCF files. The aggregate file 650 may include an aggregate number of entire reads in a bin. The aggregate number of entire reads may be determined or read from the BAM file. The aggregate file 650 may include an aggregate count for each of the nucleotide bases (e.g., A, C, T, and G) in a bin. For example, the count for each of the nucleotide bases may be determined or read from the FASTA or FASTQ file, or the BAM file. The aggregate file 650 may also, or alternatively, include counts for each variant type. For example, the aggregate file 650 may include a count of a number of gains, a number of losses, a number of insertions, a number of deletions, and/or a number of translocations.

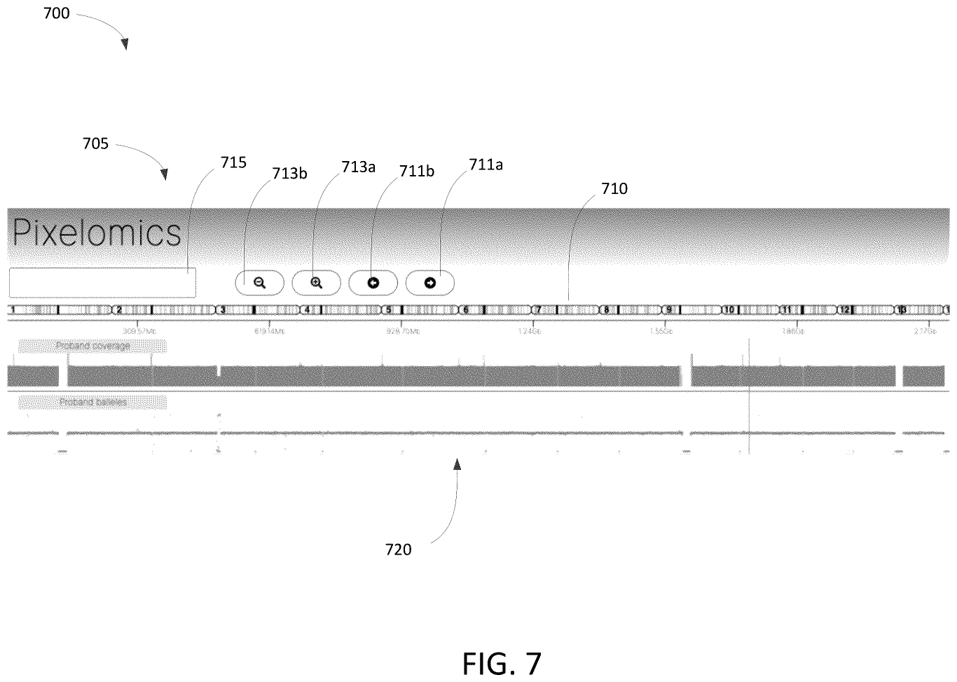

FIG. 7 depicts another example aggregate viewer 700 configured to display summary data associated with genome data. The aggregate viewer 700 may include a genome viewer or genome browser. The aggregate viewer 700 may comprise a user interface 705 that is configured to enable display and visualization of summary data associated with a genome that is stored in an aggregate file (e.g., such as the aggregate file 650 shown in FIG. 6B).

The aggregate viewer 700 may comprise a chromosome ideogram 710. The chromosome ideogram 710 may represent a view of one or more chromosomes within the genome. The aggregate viewer 700 (e.g., displayed via the user interface 705) may comprise a text box 715. The text box 715 may enable input of a genomic region (e.g., chromosome range). The text box 715 may display a selected genomic region (e.g., chromosome range). For example, the text box 715 may display the pair of genomic coordinates that defines the genomic region. In response to entry of a genomic region in the text box 715 and actuation of a button or other input from the user, the aggregate viewer 700 may send a request for the summary data for the defined genomic region. The user may zoom in or out of different portions of the genome by selecting the zoom in button 713a or the zoom out button 713b, respectively. The aggregate viewer 700 may zoom in or out by a predefined amount in response to selection of the zoom buttons 713a, 713b. The user may scroll to earlier or later genomic regions by selecting the scroll button 711b or the scroll button 711a, respectively. The aggregate viewer 700 may scroll by a predefined amount in response to selection of the scroll buttons 711a, 711b. In response to the selection of the zoom buttons 713a, 713b and/or the scroll buttons 711a, 711b, the aggregate viewer 700 may send a request for the summary data for the defined genomic region. The text box 715 and/or the chromosome ideogram 710 may be updated to indicate the defined genomic region in response to the selection of the zoom buttons 713a, 713b and/or the selection of the scroll buttons 711a, 711b.

The aggregate viewer 700 (e.g., the user interface 705) may comprise a selection display area 720. The selection display area 720 may display summary data associated with the selected portion of the genome. For example, the selection display area 720 may display summary data for a plurality of bins (e.g., at a target depth) that overlap the selected portion of the genome.

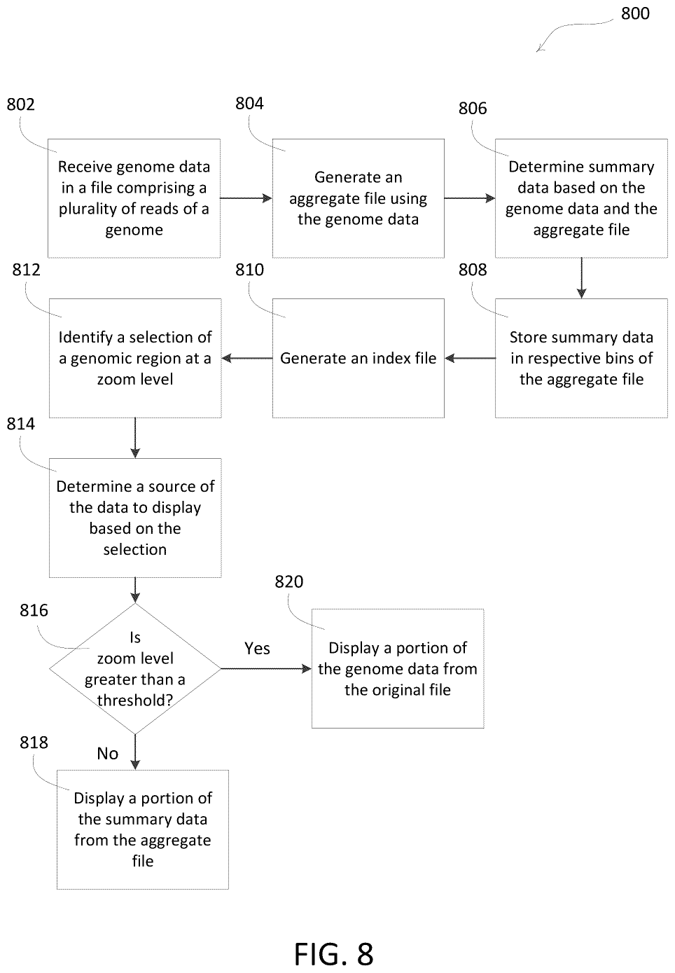

FIG. 8 is a flowchart depicting an example process 800 for generating an aggregate file and/or an index file for displaying data associated with a selected genomic region. The process 800 may enable displaying relevant data associated with a selected portion of a genome. For example, the process 800 may be used to display original data or summary data associated with the selected portion of a genome. One or more portions of the process 800 may be performed by one or more computing devices (e.g., such as the client device 108, the server device 102, and/or the computing device 200 shown in FIGS. 1A and 2, respectively). One or more portions of the process 800 may be stored in memory as computer-readable or machine-readable instructions that may be executed by a processor of the one or more computing devices. Though portions of the process 800 may be described herein as being performed by a single computing device, the process 800, or portions thereof, may be distributed across multiple devices, such as a client computing device (e.g., such as the client device 108 shown in FIG. 1A), a genotyping device (e.g., such as the sequencing device 114 shown in FIG. 1A), and/or one or more server computing devices (e.g., such as the server device(s) 102 shown in FIG. 1A).

The process 800 may begin at 802. As shown in FIG. 8, at 802 the computing device may receive genome data associated with a genome. For example, the genome data may comprise genome sequencing data. The genome data may be received in a FASTA or FASTQ file, a BED or a BedGraph file, and/or a VCF or gVCF file. The genome data may include sequencing data for a plurality of reads.

At 804, the computing device may generate an aggregate file (e.g., such as the aggregate file 650 shown in FIG. 6B) using the received genome data. The computing device may analyze the FASTA or FASTQ file, the BED file or BedGraph file, and/or a VCF or gVCF file, when generating the aggregate file. The aggregate file may comprise a plurality of nodes at a plurality of depths. Each of the plurality of nodes may represent a vertex in a graph data structure. For example, the aggregate file comprises a tree format where each of the plurality of nodes may represent a summary (e.g., summary data) of a portion of the genome data (e.g., the portion of the tree format that branches from a respective node). Each of the plurality of nodes may represent a respective bin of a plurality of bins (e.g., such as the bins 652, 654, 656, 658 shown in FIG. 6B) in the aggregate file. The plurality of bins may represent the nodes when written to the aggregate file. The plurality of nodes may be used at runtime for the aggregate file. Each of the plurality of bins may be associated with a subset of the reads in the genome data. The computing device may read the BED file or the BedGraph file to identify the plurality of reads. A read that overlaps two of the plurality of bins may be assigned to one of the two bins based on how much it overlaps each of the two bins. The plurality of bins may comprise a first set of bins at a first depth, a second set of bins at a second depth, and a third set of bins at a third depth. Each of the second set of bins may comprise a plurality of the first set of bins at the first depth. Each of the third set of bins may comprise a plurality of the second set of bins at the second depth. The VCF and/or gVCF file may be analyzed to determine variant calling information. The FASTA and/or FASTQ file may also, or alternatively, be analyzed to identify reads.

The aggregate file may be associated with coordinates for each of the plurality of bins. The coordinates may correspond to respective positions in the genome. Each of the plurality of bins in the aggregate file may comprise a begin coordinate and an end coordinate. The begin coordinate and the end coordinate may indicate the portion of the genome that is represented by the respective bin. Each of the plurality of bins may comprise the mean, min, median, max, std deviation, aggregate pointer, and/or datacount. When the aggregate file is queried, the data (e.g., the mean, min, median, max, std deviation, aggregate pointer, datacount, aggregate count, and/or aggregate count per nucleotide base) may be converted to a string format. The string format may be displayed on a command line and/or returned from an application programming interface (API) call.

At 806, the computing device may determine summary data for respective reads associated with a respective portion of the genome that each of the plurality of bins covers based on the received genome data and the aggregate file. The summary data may comprise a mean, a median, a maximum, a minimum, a standard deviation, an aggregate count, and/or an aggregate count per nucleotide base associated with the reads between the begin coordinate and the end coordinate. The summary data may comprise one or more of an average quality, an average depth, or one or more nucleotide proportions. For example the average quality may represent a mean mapping quality for the reads associated with the respective portion of the genome. The average depth may represent a mean of mapped read depths for the reads associated with the respective portion of the genome. The one or more nucleotide proportions may represent how many A bases, T bases, C bases, and G bases are within the reads associated with the respective portion of the genome. The computing device may read (e.g., analyze) the BED file to identify the respective reads when determining the summary data. For example, the computing device may analyze the reads associated with the respective portions of the genome to calculate the summary data for each of the plurality of bins. The VCF and/or gVCF file may be analyzed to determine variant calling information. The FASTA and/or FASTQ file may also, or alternatively, be analyzed to identify reads.

At 808, the computing device may store the summary data for the reads in the respective bins of the plurality of bins of the aggregate file. Each of the bins at a specific depth may comprise summary data of an equal portion of the genome. Each of the plurality of bins may occupy an equal sized space of memory. The space of memory that is occupied by each of the plurality of bins may depend on a number of discrete variables comprised within the summary data.

At 810, the computing device may generate an index file. The index file may comprise pointers to respective bins of the plurality of bins for a plurality of zoom levels at a plurality of genomic regions. The index file may comprise a plurality of depth variables and a depth offset for each of the plurality of depth variables. In another example, the computing device may forego the use of the index file and may directly access the bins based on the begin and end positions for the bin.

At 812, the computing device may identify a selection of a genomic region at a zoom level of the plurality of zoom levels. For example, the computing device may receive the selection of the genomic region at the zoom level.

At 814, the computing device may determine a source of the data for display based on the selection at 812. For example, the computing device may determine, using the index file, whether to display summary data from the aggregate file or genome data from an original file, such as the FASTA or FASTQ file, BED or BedGraph file, the VCF or gVCF file, and/or the BAM file.

At 816, the computing device may determine whether a zoom level associated with the selection at 812 is greater than a predetermined zoom threshold. The zoom level associated with the selection at 812 may meet the predetermined zoom threshold when the zoom level is greater than the predetermined zoom threshold. The zoom level associated with the selection at 812 may not meet the predetermined zoom threshold when the zoom level is less than or equal to the predetermined zoom threshold. For example, the computing device may compare at 816 the zoom level associated with the selection at 812 against the predetermined zoom threshold. The predetermined zoom threshold may be associated with an amount of genomic data from the original file (e.g., the FASTA or FASTQ file, BED or BedGraph file, the VCF or gVCF file, and/or the BAM file) that can be displayed at the same time. For example, the predetermined zoom threshold may indicate a zoom level for which the genomic data from the original file can be fully displayed. The zoom level may be determined by a predefined chromosome coordinate range.

The predetermined zoom threshold may depend on the type of genome data. For example, the predetermined zoom threshold may be adjusted based on how many data points there are in the genome. For a BED file that has a data point for every position in the genome, the predetermined zoom threshold may be set lower such that the aggregate viewer can go down to a depth that has more, smaller bins. If a BED file comprises a data point roughly every 1000 bases (e.g., how frequently a single nucleotide variant occurs), the aggregate viewer may not have to go deeper than a depth of 12. If a BED file comprises a data point every position, then the smallest bins would summarize a million data points each (e.g., rather than something more reasonable like 1000 data points).

When the zoom level associated with the selection at 812 is less than or equal to the predetermined zoom threshold, the computing device may display at 818 a portion of the summary data from the aggregate file that is associated with the selected genomic region. For example, the computing device may perform a range request on the portion of the summary data in the aggregate file that is associated with the selected genomic region. The computing device may display the portion of the summary data in a genome viewer (e.g., such as the aggregate viewer 700 shown in FIG. 7).

When the zoom level associated with the selection at 812 is greater than the predetermined zoom threshold, the computing device may display at 820 a portion of the genome data from the BED file that is associated with the selected genomic region. For example, the computing device may perform a range request on the portion of the genome data in the original file (e.g., the FASTA or FASTQ file, BED or BedGraph file, the VCF or gVCF file, and/or the BAM file) that is associated with the selected genomic region. The portion of the genome data from the original file displayed at 820 may correspond to the selected genomic region. For example, the portion of the genome data from the BED file displayed at 820 may include an average depth, an average quality, and/or nucleotide base data (e.g., nucleotide proportions) for the reads that overlap the selected genomic region. The computing device may display the portion of the summary data in a genome viewer (e.g., such as the aggregate viewer 700 shown in FIG. 7).

In addition to what has been described herein, the methods and systems may also be implemented in a computer program(s), software, or firmware incorporated in one or more computer-readable media for execution by a computer(s) or processor(s), for example. Examples of computer-readable media include electronic signals (transmitted over wired or wireless connections) and tangible/non-transitory computer-readable storage media. Examples of tangible/non-transitory computer-readable storage media include, but are not limited to, a read only memory (ROM), a random-access memory (RAM), removable disks, and optical media such as CD-ROM disks, and digital versatile disks (DVDs).

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure.

What is claimed is:

1. A method comprising:
receiving genome data associated with a genome;
generating an aggregate file using the received genome data, wherein the aggregate file comprises a plurality of bins at a plurality of depths, and wherein the plurality of bins comprises a first set of bins at a first depth, a second set of bins at a second depth, and a third set of bins at a third depth, wherein a bin of the first set of bins comprises a plurality of bins of the second set of bins at the second depth, and wherein a bin of the second set of bins comprises a plurality of bins of the third set of bins at the third depth;
determining summary data for respective reads associated with respective portions of the genome covered by respective bins of the plurality of bins, based on the received genome data and the aggregate file;
storing the summary data for the respective reads in the respective bins of the plurality of bins that cover the respective portions of the genome associated with the respective reads, wherein the second set of bins comprises summary data associated with a plurality of the first set of bins at the first depth, and wherein the third set of bins comprises summary data associated with a plurality of the second set of bins at the second depth; and
displaying a portion of the summary data in response to a selection of a genomic region by a user, wherein the displayed portion of summary data is associated with one or more of the bins of the plurality of bins that correspond with the genomic region selected by the user, and wherein the portion of summary data is displayed using one or more display conditions to represent relative differences in the summary data between the one or more bins of the displayed portion of summary data.

2. The method of claim 1, wherein the aggregate file comprises a header that indicates one or more of a name length, a genome name, a reference length, or a scale factor.

3. The method of claim 2, wherein the scale factor indicates how many bins of a proximate depth are comprised within a respective one of the plurality of bins.

4. The method of claim 2, wherein the scale factor indicates how many bins of a lower depth are combined into a respective one of the plurality of bins at a next higher depth.

5. The method of claim 2, wherein the scale factor indicates how many bins of the second set of bins are comprised within the third set of bins and how many bins of the first set of bins are comprised within the second set of bins.

6. The method of claim 2, wherein the name length and the genome name identify the genome.

7. The method of claim 2, further comprising determining a minimum depth and a maximum depth for the aggregate file based on the reference length and the scale factor.

8. The method of claim 1, wherein the summary data comprises one or more of an average quality, an average depth, or one or more nucleotide proportions.

9. The method of claim 1, further comprising identifying a location in the aggregate file that corresponds to the genomic region selected by the user.

10. The method of claim 8, wherein the location in the aggregate file comprises a specific bin of the plurality of bins at a specific depth of the plurality of depths.

11. The method of claim 1, wherein each of the plurality of bins occupies an equal sized space of memory.

12. The method of claim 1, wherein each of the bins at a specific depth comprise summary data of an equal portion of the genome.

13. The method of claim 1, wherein a read that overlaps two of the plurality of bins is assigned to one of the two bins based on how much the read overlaps each of the two bins.

14. The method of claim 1, wherein the displayed portion of summary data corresponds to a depth of the plurality of depths, the method further comprising determining the depth for the displayed portion of summary data based on the genomic region selected by the user.

15. The method of claim 14, the method further comprising identifying one or more bins at the determined depth that overlap the genomic region selected by the user.

16. The method of claim 1, wherein the one or more display conditions comprise one or more of color, opacity, or height.

17. The method of claim 1, wherein the genome data is received in an alignment map file.

18. The method of claim 17, wherein the alignment map file is a binary alignment map (BAM) file or a sequence alignment map (SAM) file.

19. The method of claim 18, further comprising reading the BAM file to identify the respective reads.

20. A method comprising:
receiving genome data associated with a genome in a browser extensible data (BED) file;
generating an aggregate file using the received genome data, wherein the aggregate file comprises a plurality of bins at a plurality of depths, wherein bins of the plurality of bins represent respective portions of the genome;
determining summary data for respective reads associated with a respective portion of the genome for one or more bins of the plurality of bins based on the received genome data and the aggregate file;
storing the summary data for the reads in the respective bins of the plurality of bins of the aggregate file;
generating an index file that comprises pointers to respective bins of the plurality of bins for a plurality of zoom levels at a plurality of genomic regions;
identifying a selection of a genomic region of the plurality of genomic regions at a zoom level of the plurality of zoom levels;
determining, using the index file, whether to display summary data from the aggregate file or genome data from the BED file; and
displaying, based on the determination, a portion of the summary data that corresponds with the selection of the genomic region by a user, wherein the portion of summary data is displayed using one or more display conditions to represent relative differences in the summary data between the one or more bins of the displayed portion of summary data.

21. The method of claim 20, wherein each of the plurality of bins in the aggregate file comprises a string that indicates a begin location and an end location for the respective node.

22. The method of claim 20, wherein it is determined to display a portion of the genome data from the BED file when the zoom level is greater than a predetermined zoom threshold.

23. The method of claim 22, further comprising performing a range request on the portion of the genome data in the BED file associated with the selected genomic region.

24. The method of claim 20, wherein it is determined to display the portion of the summary data from the aggregate file when the zoom level is less than or equal to the predetermined zoom threshold.

25. The method of claim 24, further comprising performing a range request on the portion of the summary data in the aggregate file associated with the selected genomic region.

26. The method of claim 20, wherein the summary data comprises one or more of an average quality, an average depth, or one or more nucleotide proportions.

27. The method of claim 20, wherein the index file indicates a node size for each depth of the plurality of depths.

28. The method of claim 20, wherein the aggregate file comprises coordinates for each of the plurality of bins.

29. The method of claim 28, wherein the coordinates correspond to respective positions in the genome.

30. The method of claim 20, wherein identifying the selection of the genomic region comprises receiving the selection of the genomic region.

31. The method of claim 20, wherein the aggregate file comprises a tree format.

32. The method of claim 20, further comprising reading the BED file to identify the respective reads.

33. The method of claim 20, wherein the one or more display conditions comprise one or more of color, opacity, or height.

34. The method of claim 20, wherein the index file comprises a plurality of depth variables having respective depth offsets.

35. The method of claim 20, wherein the displayed portion of summary data is retrieved from the aggregate file.

36. The method of claim 20, wherein the displayed portion of summary data is retrieved from a portion of the genome data from the BED file.
